(19) United States
(12) Patent Application Publication
(10) Pub. No.: US 2022/0235335 A1
(43) Pub. Date: Jul. 28, 2022

(54) Glycosyltransferase Mutant and Use Therefor

(71) Applicant: CAS Center for Excellence in Molecular Plant Sciences, Shanghai (CN)

(72) Inventors: Yong Wang, Shanghai (CN); Zhifeng Liu, Shanghai (CN); Yuwei Sun, Shanghai (CN); Huajun Lv, Shanghai (CN); Peng Zhang, Shanghai (CN); Jianxu Li, Shanghai (CN); Haili Liu, Shanghai (CN); Jianhua Li, Shanghai (CN); Zhuo Chen, Shanghai (CN)

(21) Appl. No.: 17/618,665

(22) PCT Filed: Jul. 27, 2020
(86) PCT No.: PCT/CN2020/104957

§ 371 Date: Dec. 13, 2021

(30) Foreign Application Priority Data
Jun. 14, 2019 (CN) 201910515613.1
Sep. 26, 2019 (CN) 201910917940.X

(51) Int. Cl. C12N 9/10; C12N 15/63; C12Q 1/686; C12P 19/56

(52) U.S. Cl. CPC C12N 9/1048; C12P 19/56; C12Q 1/686; C12N 15/63

(57) ABSTRACT

Provided are a mutant glycosyltransferase UGT76G1 and a use therefor, the catalytic activity, the substrate selectivity, and/or the substrate specificity of the mutant glycosyltransferase UGT76G1 having been changed. Mutation at specific points can promote the catalytic activity for 1,3-glycosylation of a substrate containing 1,2-diglucosyl (sophorosyl), and weaken the catalytic activity thereof to perform 1,3-glycosylation on a glucose monosaccharide substrate base. Also provided are mutations that weaken the catalytic activity of glycosyltransferase UGT76G1, able to increase accumulation of a specific stevioside intermediate.

### TECHNICAL FIELD

The invention belongs to the field of biotechnology. More specifically, the invention relates to a glycosyltransferase mutant and use therefor.

### BACKGROUND OF DISCLOSURE

Glycosylation is one of the most extensive modifications in the synthesis of natural products. In plants, glycosylation changes the solubility, stability, toxicity and physiological activity of natural products. It has the functions of metabolite detoxification, preventing biological invasion, changing the distribution range of substances and so on. The glycosylation of many natural products from plants is catalyzed by UDP dependent glycosyltransferase (UGT), which uses UDP activated sugars as glycosyl donors to specifically transfer glycans to the glycosylation sites of receptor molecules. At present, more than 2300 UGTs from plants have been found or annotated, but only about 20 UGTs have been analyzed for protein structure.

Stevia glycosides are a kind of highly glycosylated diterpene natural products, mainly from Stevia rebaudiana (Compositae plants). Stevia glycosides have high sweetness and low calorie. They can replace sucrose and other artificial sweeteners, therefore have great economic benefits in the food industry. At present, the widely used Stevia sugars mainly include natural rebaudioside A and stevioside. Although these products have sweetness 300 to 600 times than that of sucrose, they still have disadvantages such as bitter aftertaste, and the taste needs to be improved. In recent years, the industrial improvement of Stevia sugars mainly focuses on upgrading rebaudioside A and stevioside to rebaudioside D and rebaudioside M with better taste and higher sweetness.

The content of rebaudioside D and rebaudioside M in the original plants is very low. The cost of extracting and purifying them from plants is huge, and the current output is far from being able to meet the market demand. Rebaudioside D and rebaudioside M are polyglycosides formed by the aglycon steviol (steviol) through 5-step and 6-step glycosylation, respectively. The intermediates in their synthesis pathways include rebaudioside A and stevioside. It is reported that UGT76G1 is responsible for catalyzing stevioside to convert to rebaudioside A. Rebaudioside A is catalyzed by UGT91D2 (or EUGT11) to form rebaudioside D, or catalyzed by UGT76G1 to form by-product rebaudioside I. Rebaudioside D is further catalyzed by UGT76G1 to produce rebaudioside M. Therefore, UGT76G1 and UGT91D2 are the two key enzyme genes required for repeated glycosylation during the synthesis of Rebaudioside D and Rebaudioside M.

Because the glycosyltransferase UGT76G1 participates in several glycosylation steps during the synthesis of steviol glycosides, there are problems such as poor substrate specificity and weak catalytic activity. There is an urgent need in this field to explore methods for improving the substrate specificity and catalytic activity of UGT76G1.

### SUMMARY OF DISCLOSURE

The purpose of the present invention is to provide a glycosyltransferase mutant and use thereof.

In the first aspect of the present invention, a glycosyltransferase UGT76G1 mutant is provided, which has a mutation in the amino acid interacting with the glycosyl donor or glycosyl receptor in its spatial structure and has changes in its catalytic activity, as compared to the wild-type glycosyltransferase UGT76G1.

In a preferred embodiment, the catalytic activity to convert substrate rebaudioside D to rebaudioside M is statistically significantly increased, such as by more than 20%, more than 40%, more than 60%, more than 70% or higher.

In another preferred embodiment, the catalytic activity to convert rebaudioside A to by-product rebaudioside I is statistically significantly decreased, such as by more than 20%, more than 40%, more than 50% or higher.

In another preferred embodiment, the glycosyltransferase UGT76G1 mutant is:

(a) a protein of amino acid sequence corresponds to SEQ ID NO: 1, with a mutation at residue 284, 147, 155, 146, 380, 85, 87, 88, 90, 91, 126, 196, 199, 200, 203, 204 or 379;

(b) a protein derived from (a) having one or more (such as 1-20; preferably 1-15; more preferably 1-10, such as 5, 3) amino acids substituted, deleted, or inserted in the sequence, and still having the function of the protein of (a), while the amino acids corresponding to residue 284, 147, 155, 146, 380, 85, 87, 88, 90, 91, 126, 196, 199, 200, 203, 204 or 379 of SEQ ID NO: 1 are the same as those mutated at the corresponding position of the protein of (a);

(c) a protein derived from (a) having more than 80% (preferably more than 85%; more preferably more than 90%; more preferably more than 95%, such as 98%, 99%) sequence identity with the amino acid sequence of the protein of (a), and having the function of the protein of (a), while the amino acids corresponding to residue 284, 147, 155, 146, 380, 85, 87, 88, 90, 91, 126, 196, 199, 200, 203, 204 or 379 of SEQ ID NO: 1 are the same as those mutated at the corresponding position of the protein of (a);

(d) the active fragment of the protein of (a), which contains the structure interacting with the glycosyl donor or glycosyl receptor in the spatial structure of glycosyltransferase UGT76G1, the amino acids corresponding to residue 284, 147, 155, 146, 380, 85, 87, 88, 90, 91, 126, 196, 199, 200, 203, 204 or 379 of SEQ ID NO: 1 are the same as those mutated at the corresponding position of the protein of (a).

In another preferred embodiment, in the glycosyltransferase UGT76G1 mutant, the 284th residue is mutated to Ser. The catalytic activity of the mutant is improved, preferably, its catalytic activity for 1,3-glycosylation of a substrate containing 1,2-diglucosyl is increased or its catalytic activity for 1,3-glycosylation based on a monoglucosyl substrate is reduced; preferably, its catalytic activity for the substrate steviolbioside, stevioside or rebaudioside D is increased, while its catalytic activity for the substrate steviolmonoside, rubusoside and rebaudioside A is reduced; more preferably, its catalytic activity to convert rebaudioside D to rebaudioside M is increased and its catalytic activity to convert rebaudioside A to by-product rebaudioside I is decreased.

In another preferred embodiment, the 284th residue of the glycosyltransferase UGT76G1 mutant is mutated to Ala, and the catalytic activity of the mutant is decreased.

In another preferred embodiment, the 147th residue of the glycosyltransferase UGT76G1 mutant is mutated to Ala, Asn or Gln, and the catalytic activity of the mutant is decreased.

In another preferred embodiment, the 155th residue of the glycosyltransferase UGT76G1 mutant is mutated to Ala or Tyr, and the catalytic activity of the mutant is decreased.

In another preferred embodiment, the 146th residue of the glycosyltransferase UGT76G1 mutant is mutated to Ala, Asn or Ser, and the catalytic activity of the mutant is decreased.

In another preferred embodiment, the 380th residue of the glycosyltransferase UGT76G1 mutant is mutated to Thr, Ser, Asn or Glu, and the catalytic activity of the mutant is decreased or lost.

In another preferred embodiment, the 85th residue of the glycosyltransferase UGT76G1 mutant is mutated to Val, and the catalytic activity of the mutant for steviolmonoside, steviolbioside, rubusoside or rebaudioside D is increased.

In another preferred embodiment, the 87th residue of the glycosyltransferase UGT76G1 mutant is mutated to Phe, and the catalytic activity of the mutant for steviolmonoside, steviolbioside, rubusoside, stevioside, rebaudioside A or rebaudioside D is decreased.

In another preferred embodiment, the 88th residue of the glycosyltransferase UGT76G1 mutant is mutated to Val, and the catalytic activity of the mutant for steviolbioside, stevioside, rebaudioside A or rebaudioside D is increased; the catalytic activity for steviolmonoside is decreased.

In another preferred embodiment, the 90th residue of the glycosyltransferase UGT76G1 mutant is mutated to Leu, and the catalytic activity of the mutant for steviolbioside is increased;

the catalytic activity for steviolmonoside or rubusoside is decreased.

In another preferred embodiment, the 90th residue of the glycosyltransferase UGT76G1 mutant is mutated to Val, and the catalytic activity of the mutant for steviolbioside or stevioside is increased; the catalytic activity for steviolmonoside or rubusoside is decreased.

In another preferred embodiment, the 91th residue of the glycosyltransferase UGT76G1 mutant is mutated to Phe, and the catalytic activity of the mutant for steviolbioside is increased; the catalytic activity for steviolmonoside, rubusoside or stevioside is decreased.

In another preferred embodiment, the 126th residue of the glycosyltransferase UGT76G1 mutant is mutated to Phe, and the catalytic activity of the mutant for steviolbioside, stevioside or rebaudioside D is increased; the catalytic activity for steviolmonoside, rubusoside or rebaudioside A is decreased.

In another preferred embodiment, the 126th residue of the glycosyltransferase UGT76G1 mutant is mutated to Val, and the catalytic activity of the mutant for steviolmonoside, rubusoside, stevioside or rebaudioside A is decreased.

In another preferred embodiment, the 196th residue of the glycosyltransferase UGT76G1 mutant is mutated to Gln, and the catalytic activity of the mutant for steviolmonoside or rebaudioside D is decreased.

In another preferred embodiment, the 199th residue of the glycosyltransferase UGT76G1 mutant is mutated to Phe, and the catalytic activity of the mutant for steviolmonoside, steviolbioside or rebaudioside D is increased.

In another preferred embodiment, the 199th residue of the glycosyltransferase UGT76G1 mutant is mutated to Leu, and the catalytic activity of the mutant for steviolmonoside, steviolbioside, rubusoside or rebaudioside D is increased.

In another preferred embodiment, the 199th residue of the glycosyltransferase UGT76G1 mutant is mutated to Val, and the catalytic activity of the mutant for steviolbioside, stevioside, rebaudioside A or rebaudioside D is increased.

In another preferred embodiment, the 200th residue of the glycosyltransferase UGT76G1 mutant is mutated to Ile, and the catalytic activity of the mutant for steviolbioside, rebaudioside A or rebaudioside D is increased; the catalytic activity for steviolmonoside or rubusoside is decreased.

In another preferred embodiment, the 200th residue of the glycosyltransferase UGT76G1 mutant is mutated to Val, and the catalytic activity of the mutant for rebaudioside A is increased; the catalytic activity for steviolmonoside or rubusoside is decreased.

In another preferred embodiment, the 203th residue of the glycosyltransferase UGT76G1 mutant is mutated to Leu, and the catalytic activity of the mutant for steviolmonoside, rubusoside, rebaudioside A or rebaudioside D is decreased.

In another preferred embodiment, the 203th residue of the glycosyltransferase UGT76G1 mutant is mutated to Val, and the catalytic activity of the mutant for steviolbioside or rebaudioside D is increased; the catalytic activity for steviolmonoside, rubusoside or rebaudioside A is decreased.

In another preferred embodiment, the 204th residue of the glycosyltransferase UGT76G1 mutant is mutated to Phe, and the catalytic activity of the mutant for steviolmonoside, rubusoside, stevioside or rebaudioside D is decreased.

In another preferred embodiment, the 204th residue of the glycosyltransferase UGT76G1 mutant is mutated to Trp, and the catalytic activity of the mutant for steviolmonoside, steviolbioside, rubusoside, stevioside, rebaudioside A or rebaudioside D is decreased.

In another preferred embodiment, the 379th residue of the glycosyltransferase UGT76G1 mutant is mutated to Phe, and the catalytic activity of the mutant for steviolbioside is increased; the catalytic activity for steviolmonoside, rubusoside, stevioside or rebaudioside D is decreased.

In another preferred embodiment, the 379th residue of the glycosyltransferase UGT76G1 mutant is mutated to Ile, and the catalytic activity of the mutant for steviolmonoside, steviolbioside, stevioside, rebaudioside A or rebaudioside D is increased.

In another preferred embodiment, the 379th residue of the glycosyltransferase UGT76G1 mutant is mutated to Val, and the catalytic activity of the mutant for steviolbioside, rebaudioside A or rebaudioside D is increased; the catalytic activity for steviolmonoside, rubusoside or stevioside is decreased.

In another preferred embodiment, the 379th residue of the glycosyltransferase UGT76G1 mutant is mutated to Trp, and the catalytic activity of the mutant for rebaudioside A is increased; the catalytic activity for steviolbioside is decreased.

In another preferred embodiment, the 199th, 200th, and 203th residues of the glycosyltransferase UGT76G1 mutant are mutated to Ala, and the catalytic activity of the mutant for rebaudioside A is increased; the catalytic activity for steviolmonoside, steviolbioside, rubusoside or stevioside is decreased.

In another preferred embodiment, the 199th, 200th, 203th and 204th residues of the glycosyltransferase UGT76G1 mutant are mutated to Ala, and the catalytic activity of the mutant for steviolmonoside, steviolbioside, rubusoside, stevioside or rebaudioside D is decreased.

In another aspect of the present disclosure, an isolated polynucleotide encoding the above glycosyltransferase UGT76G1 mutant is provided.

In another aspect of the present disclosure, a vector is provided, which contains the polynucleotide.

In another aspect of the present disclosure, a genetically engineered host cell is provided, which contains the vector or has the polynucleotide integrated in the genome.

In a preferred embodiment, the cell comprises: a reaction system for 1,3-glycosylation based on 1,2-diglucosyl or monoglucosyl substrate, wherein the enzyme for glycosylation (including 1,3-glycosylation of 1,2-diglucosyl or monoglucosyl substrate) is a glycosyltransferase UGT76G1 mutant; preferably, the reaction system is a system for rebaudioside M production.

In another preferred embodiment, the system for rebaudioside M production comprises a system with rebaudioside A as a substrate, including a glycosyltransferase UGT76G1 mutant with residue 284 mutated to Ser, residue 85 mutated to Val, residue 126 mutated to Phe, residue 199 mutated to Phe, residue 199 mutated to Leu or residue 203 mutated to Val, corresponding to SEQ ID NO: 1, and an enzyme for converting rebaudioside A into rebaudioside D; preferably, the enzyme for converting rebaudioside A into rebaudioside D includes (but is not limited to): EUGT11, UGT91D2.

In another preferred embodiment, the system for rebaudioside M production comprises a system with stevioside as a substrate, including an enzyme for converting stevioside to rebaudioside A, a glycosyltransferase UGT76G1 mutant with residue 284 mutated to Ser, residue 88 mutated to Val, residue 90 mutated to Val, residue 126 mutated to Phe, residue 199 mutated to Val, or residue 379 mutated to Ile, corresponding to SEQ ID NO: 1, and an enzyme for converting rebaudioside A into rebaudioside D; preferably, the enzyme for converting stevioside to rebaudioside A is also UGT76G1, UGT76G1 mutant, the enzyme for converting rebaudioside A into rebaudioside D includes (but is not limited to): EUGT11, UGT91D2.

In another preferred embodiment, the system for rebaudioside M production comprises a system with rebaudioside D as a substrate, including a glycosyltransferase UGT76G1 mutant with residue 284 mutated to Ser, residue 85 mutated to Val, residue 88 mutated to Val, residue 126 mutated to Phe, residue 199 mutated to Phe, residue 199 mutated to Leu, residue 199 mutated to Val, residue 200 mutated to Ile, residue 203 mutated to Val, residue 379 mutated to Ile, residue 379 mutated to Val, or residue 379 mutated to Trp, corresponding to SEQ ID NO: 1.

In another preferred embodiment, the system for rebaudioside M production comprises a system with steviol as a substrate, including a glycosyltransferase UGT76G1 mutant with residue 284 mutated to Ser, residue 88 mutated to Val, residue 90 mutated to Val, residue 126 mutated to Phe, residue 199 mutated to Val, or residue 379 mutated to Ile, corresponding to SEQ ID NO: 1, and an enzyme for converting rebaudioside A or stevioside into rebaudioside D and an enzyme for converting steviol into stevioside or rebaudioside A; the enzyme for converting steviol into stevioside or rebaudioside A or includes (but is not limited to): EUGT11, UGT91D2, UGT74G1, UGT85C2, UGT75L20, UGT75L21, UGT75W2, UGT75T4, UGT85A57, UGT85A58, UGT76G1, UGT76G1 mutant.

In another preferred embodiment, the host cell also includes an enzyme for recycling UDP glucose; preferably, the enzyme for recycling UDP glucose includes (but is not limited to): AtSUS3.

In another preferred embodiment, the host cell may include a prokaryotic cell or a eukaryotic cell; preferably, the prokaryotic cell includes Escherichia coli or Bacillus subtilis, or the eukaryotic cell includes a fungal cell, a yeast cell, an insect cell or a mammalian cell.

Another aspect of the present disclosure provides a method for producing the glycosyltransferase UGT76G1 mutant of any embodiment above, comprising the steps of: (1) culturing the host cell to obtain a culture; and (2) isolating the glycosyltransferase UGT76G1 mutant from the culture.

Another aspect of the present disclosure provides a method for regulating the catalytic activity or substrate specificity of glycosyltransferase UGT76G1, including: mutating the amino acid interacting with glycosyl donor or glycosyl receptor in its spatial structure; thereby changing its catalytic activity or substrate specificity.

In a preferred embodiment, mutating the 284th residue corresponding to SEQ ID NO:1 to Ser, and the catalytic activity of the mutant for 1,3-glycosylation of a substrate containing 1,2-diglucosyl (such as steviolbioside, stevioside or rebaudioside D) is increased or its catalytic activity for 1,3-glycosylation based on a monoglucosyl substrate (such as steviolmonoside, rubusoside, rebaudioside A) is reduced; preferably, its catalytic activity to convert rebaudioside D to rebaudioside M is increased and its catalytic activity to convert rebaudioside A to by-product rebaudioside I is decreased; or mutating the 284th residue corresponding to SEQ ID NO: 1 to Ala to decrease the catalytic activity of the mutant; or mutating the 147th residue corresponding to SEQ ID NO: 1 to Ala, Asn or Gln to decrease the catalytic activity of the mutant; or mutating the 155th residue corresponding to SEQ ID NO: 1 to Ala or Tyr to decrease the catalytic activity of the mutant; or mutating the 146th residue corresponding to SEQ ID NO: 1 to Ala, Asn or Ser to decrease the catalytic activity of the mutant; or mutating the 380th residue corresponding to SEQ ID NO: 1 to Thr, Ser, Asn or Glu to decrease the catalytic activity of the mutant or eliminate the catalytic activity. In another preferred embodiment, the method including: mutating the 85th residue corresponding to SEQ ID NO:1 to Val, and the catalytic activity of the mutant for steviolmonoside, steviolbioside, rubusoside or rebaudioside D is increased. mutating the 87th residue corresponding to SEQ ID NO:1 to Phe, and the catalytic activity of the mutant for steviolmonoside, steviolbioside, rubusoside, stevioside, rebaudioside A or rebaudioside D is decreased; mutating the 88th residue corresponding to SEQ ID NO:1 to Val, and the catalytic activity of the mutant for substrate steviolbioside, stevioside, rebaudioside A or rebaudioside D is increased, and its catalytic activity for substrate steviolmonoside is decreased; mutating the 90th residue corresponding to SEQ ID NO:1 to Leu, and the catalytic activity of the mutant for substrate steviolbioside is increased, and its catalytic activity for substrate steviolmonoside, or rubusoside is decreased; mutating the 90th residue corresponding to SEQ ID NO:1 to Val, and the catalytic activity of the mutant for substrate steviolbioside or stevioside is increased, and its catalytic activity for substrate steviolmonoside, or rubusoside is decreased; mutating the 91th residue corresponding to SEQ ID NO:1 to Phe, and the catalytic activity of the mutant for substrate steviolbioside is increased, and its catalytic activity for substrate steviolmonoside, rubusoside, or stevioside is decreased; mutating the 126th residue corresponding to SEQ ID NO:1 to Phe, and the catalytic activity of the mutant for substrate steviolbioside, stevioside or rebaudioside D is increased, and its catalytic activity for substrate steviolmonoside, rubusoside or rebaudioside A is decreased; mutating the 126th residue corresponding to SEQ ID NO:1 to Val, and the catalytic activity of the mutant for steviolmonoside, rubusoside, stevioside, or rebaudioside A is decreased; mutating the 196th residue corresponding to SEQ ID NO:1 to Gln, and the catalytic activity of the mutant for steviolmonoside or rebaudioside D is decreased; mutating the 199th residue corresponding to SEQ ID NO:1 to Phe, and the catalytic activity of the mutant for steviolmonoside, steviolbioside or rebaudioside D is increased; mutating the 199th residue corresponding to SEQ ID NO:1 to Leu, and the catalytic activity of the mutant for steviolmonoside, steviolbioside, rubusoside or rebaudioside D is increased; mutating the 199th residue corresponding to SEQ ID NO:1 to Val, and the catalytic activity of the mutant for steviolbioside, stevioside, rebaudioside A or rebaudioside D is increased; mutating the 200th residue corresponding to SEQ ID NO:1 to Ile, and the catalytic activity of the mutant for substrate steviolbioside, rebaudioside A or rebaudioside D is increased, and its catalytic activity for substrate steviolmonoside or rubusoside is decreased; mutating the 200th residue corresponding to SEQ ID NO:1 to Val, and the catalytic activity of the mutant for substrate rebaudioside A is increased, and its catalytic activity for substrate steviolmonoside, or rubusoside is decreased; mutating the 203th residue corresponding to SEQ ID NO:1 to Leu, and the catalytic activity of the mutant for steviolmonoside, rubusoside, rebaudioside A or rebaudioside D is decreased; mutating the 203th residue corresponding to SEQ ID NO:1 to Val, and the catalytic activity of the mutant for substrate steviolbioside or rebaudioside D is increased, and its catalytic activity for substrate steviolmonoside, rubusoside or rebaudioside A is decreased; mutating the 204th residue corresponding to SEQ ID NO:1 to Phe, and the catalytic activity of the mutant for steviolmonoside, rubusoside, stevioside, or rebaudioside D is decreased; mutating the 204th residue corresponding to SEQ ID NO:1 to Trp, and the catalytic activity of the mutant for steviolmonoside, steviolbioside, rubusoside, stevioside, rebaudioside A or rebaudioside D is decreased; mutating the 379th residue corresponding to SEQ ID NO:1 to Phe, and the catalytic activity of the mutant for substrate steviolbioside is increased, and its catalytic activity for substrate steviolmonoside, rubusoside, stevioside or rebaudioside D is decreased; mutating the 379th residue corresponding to SEQ ID NO:1 to Ile, and the catalytic activity of the mutant for substrate steviolmonoside, steviolbioside, stevioside, rebaudioside A or rebaudioside D is increased; mutating the 379th residue corresponding to SEQ ID NO:1 to Val, and the catalytic activity of the mutant for substrate steviolbioside, rebaudioside A or rebaudioside D is increased, and its catalytic activity for substrate steviolmonoside, rubusoside or stevioside is decreased; mutating the 379th residue corresponding to SEQ ID NO:1 to Trp, and the catalytic activity of the mutant for substrate stevioside or rebaudioside A is increased, and its catalytic activity for substrate steviolbioside is decreased; mutating the 199th, 200th, 203th residues corresponding to SEQ ID NO:1 to Ala, and the catalytic activity of the mutant for substrate rebaudioside A is increased, and its catalytic activity for substrate steviolmonoside, steviolbioside, rubusoside or stevioside is decreased; or mutating the 199th, 200th, 203th 204th residues corresponding to SEQ ID NO:1 to Ala, and the catalytic activity of the mutant for steviolmonoside, steviolbioside, rubusoside, stevioside or rebaudioside D is decreased.

In another aspect of the present invention, use of the glycosyltransferase UGT76G1 mutant having amino acid sequence corresponds SEQ ID NO: 1 and residue 284 mutated to Ser is provided for promoting 1,3-glycosylation of a substrate containing 1,2-diglucosyl and reducing 1,3-glycosylation based on a monoglucosyl substrate; preferably, for promoting the production of rebaudioside D to rebaudioside M.

In another aspect of the invention, a method of regulating glycosylation is provided, which comprising promoting 1,3-glycosylation of a substrate containing 1,2-diglucosyl by conducting catalyzation via a glycosyltransferase UGT76G1 mutant having residue 284 mutated to Ser corresponding to SEQ ID NO: 1; conducting catalyzation via a glycosyltransferase UGT76G1 mutant having residue 284 mutated to Ala corresponding to SEQ ID NO: 1 to decrease glycosylation catalytic activity; conducting catalyzation via a glycosyltransferase UGT76G1 mutant having residue 147 mutated to Ala, Asn or Gln corresponding to SEQ ID NO: 1 to decrease glycosylation catalytic activity; conducting catalyzation via a glycosyltransferase UGT76G1 mutant having residue 155 mutated to Ala or Tyr corresponding to SEQ ID NO: 1 to decrease glycosylation catalytic activity; conducting catalyzation via a glycosyltransferase UGT76G1 mutant having residue 146 mutated to Ala, Asn or Ser corresponding to SEQ ID NO: 1 to decrease glycosylation catalytic activity; conducting catalyzation via a glycosyltransferase UGT76G1 mutant having residue 380 mutated to Thr, Ser, Asn or Glu corresponding to SEQ ID NO: 1 to decrease glycosylation catalytic activity or eliminate the catalytic activity; conducting catalyzation via a glycosyltransferase UGT76G1 mutant having residue 85 mutated to Val corresponding to SEQ ID NO: 1 to increase glycosylation catalytic activity for substrate steviolmonoside, steviolbioside, rubusoside or rebaudioside D; conducting catalyzation via a glycosyltransferase UGT76G1 mutant having residue 87 mutated to Phe corresponding to SEQ ID NO: 1 to decrease glycosylation catalytic activity for substrate steviolmonoside, steviolbioside, rubusoside, stevioside, rebaudioside A or rebaudioside D; conducting catalyzation via a glycosyltransferase UGT76G1 mutant having residue 88 mutated to Val corresponding to SEQ ID NO: 1 to increase glycosylation catalytic activity for substrate steviolbioside, stevioside, rebaudioside A or rebaudioside D; and to decrease glycosylation catalytic activity for substrate steviolmonoside; conducting catalyzation via a glycosyltransferase UGT76G1 mutant having residue 90 mutated to Leu corresponding to SEQ ID NO: 1 to increase glycosylation catalytic activity for substrate steviolbioside; and to decrease glycosylation catalytic activity for substrate steviolmonoside or rubusoside; conducting catalyzation via a glycosyltransferase UGT76G1 mutant having residue 90 mutated to Val corresponding to SEQ ID NO: 1 to increase glycosylation catalytic activity for substrate steviolbioside or stevioside; and to decrease glycosylation catalytic activity for substrate steviolmonoside or rubusoside; conducting catalyzation via a glycosyltransferase UGT76G1 mutant having residue 91 mutated to Phe corresponding to SEQ ID NO: 1 to increase glycosylation catalytic activity for substrate steviolbioside; and to decrease glycosylation catalytic activity for substrate steviolmonoside, rubusoside, or stevioside; conducting catalyzation via a glycosyltransferase UGT76G1 mutant having residue 126 mutated to Phe corresponding to SEQ ID NO: 1 to increase glycosylation catalytic activity for substrate steviolbioside, stevioside or rebaudioside D; and to decrease glycosylation catalytic activity for substrate steviolmonoside, rubusoside or rebaudioside A; conducting catalyzation via a glycosyltransferase UGT76G1 mutant having residue 126 mutated to Val corresponding to SEQ ID NO: 1 to decrease glycosylation catalytic activity for steviolmonoside, rubusoside, stevioside or rebaudioside A; conducting catalyzation via a glycosyltransferase UGT76G1 mutant having residue 196 mutated to Gln corresponding to SEQ ID NO: 1 to decrease glycosylation catalytic activity for steviolmonoside or rebaudioside D; conducting catalyzation via a glycosyltransferase UGT76G1 mutant having residue 199 mutated to Phe corresponding to SEQ ID NO: 1 to increase glycosylation catalytic activity for steviolmonoside, steviolbioside or rebaudioside D; conducting catalyzation via a glycosyltransferase UGT76G1 mutant having residue 199 mutated to Leu corresponding to SEQ ID NO: 1 to increase glycosylation catalytic activity for steviolmonoside, steviolbioside, rubusoside or rebaudioside D; conducting catalyzation via a glycosyltransferase UGT76G1 mutant having residue 199 mutated to Val corresponding to SEQ ID NO: 1 to increase glycosylation catalytic activity for steviolbioside, stevioside, rebaudioside A or rebaudioside D; conducting catalyzation via a glycosyltransferase UGT76G1 mutant having residue 200 mutated to Ile corresponding to SEQ ID NO: 1 to increase glycosylation catalytic activity for substrate steviolbioside, rebaudioside A or rebaudioside D; and to decrease glycosylation catalytic activity for substrate steviolmonoside or rubusoside; conducting catalyzation via a glycosyltransferase UGT76G1 mutant having residue 200 mutated to Val corresponding to SEQ ID NO: 1 to increase glycosylation catalytic activity for substrate rebaudioside A; and to decrease glycosylation catalytic activity for substrate steviolmonoside or rubusoside; conducting catalyzation via a glycosyltransferase UGT76G1 mutant having residue 203 mutated to Leu corresponding to SEQ ID NO: 1 to decrease glycosylation catalytic activity for substrate steviolmonoside, rubusoside, rebaudioside A or rebaudioside D; conducting catalyzation via a glycosyltransferase UGT76G1 mutant having residue 203 mutated to Val corresponding to SEQ ID NO: 1 to increase glycosylation catalytic activity for substrate steviolbioside or rebaudioside D; and to decrease glycosylation catalytic activity for substrate steviolmonoside, rubusoside or rebaudioside A; conducting catalyzation via a glycosyltransferase UGT76G1 mutant having residue 204 mutated to Phe corresponding to SEQ ID NO: 1 to decrease glycosylation catalytic activity for steviolmonoside, rubusoside, stevioside or rebaudioside D; conducting catalyzation via a glycosyltransferase UGT76G1 mutant having residue 204 mutated to Trp corresponding to SEQ ID NO: 1 to decrease glycosylation catalytic activity for steviolmonoside, steviolbioside, rubusoside, stevioside, rebaudioside A or rebaudioside D; conducting catalyzation via a glycosyltransferase UGT76G1 mutant having residue 379 mutated to Phe corresponding to SEQ ID NO: 1 to increase glycosylation catalytic activity for substrate steviolbioside; and to decrease glycosylation catalytic activity for substrate steviolmonoside, rubusoside, stevioside or rebaudioside D; conducting catalyzation via a glycosyltransferase UGT76G1 mutant having residue 379 mutated to Ile corresponding to SEQ ID NO: 1 to increase glycosylation catalytic activity for steviolmonoside, steviolbioside, stevioside, rebaudioside A or rebaudioside D; conducting catalyzation via a glycosyltransferase UGT76G1 mutant having residue 379 mutated to Val corresponding to SEQ ID NO: 1 to increase glycosylation catalytic activity for substrate steviolbioside, rebaudioside A or rebaudioside D; and to decrease glycosylation catalytic activity for substrate steviolmonoside, rubusoside or stevioside; conducting catalyzation via a glycosyltransferase UGT76G1 mutant having residue 379 mutated to Trp corresponding to SEQ ID NO: 1 to increase glycosylation catalytic activity for substrate rebaudioside A; and to decrease glycosylation catalytic activity for substrate steviolbioside; conducting catalyzation via a glycosyltransferase UGT76G1 mutant having residue 199, 200, 203 mutated to Ala corresponding to SEQ ID NO: 1 to increase glycosylation catalytic activity for substrate rebaudioside A; and to decrease glycosylation catalytic activity for substrate steviolmonoside, steviolbioside, rubusoside or stevioside; or conducting catalyzation via a glycosyltransferase UGT76G1 mutant having residue 199, 200, 203, 204 mutated to Ala corresponding to SEQ ID NO: 1 to decrease glycosylation catalytic activity for steviolmonoside, steviolbioside, rubusoside, stevioside or rebaudioside D.

In a preferred embodiment, the glycosylation product (1,3-glycosylation product) is rebaudioside M, and the method includes conducting catalyzation with rebaudioside A as substrate via a glycosyltransferase UGT76G1 mutant with residue 284 mutated to Ser, residue 85 mutated to Val, residue 126 mutated to Phe, residue 199 mutated to Phe, residue 199 mutated to Leu or residue 203 mutated to Val, corresponding to SEQ ID NO: 1, and an enzyme for converting rebaudioside A into rebaudioside D, to produce rebaudioside M; preferably, the enzyme for converting rebaudioside A into rebaudioside D includes: EUGT11, UGT91D2; or the method includes conducting catalyzation with stevioside as a substrate via an enzyme for converting stevioside to rebaudioside A, a glycosyltransferase UGT76G1 mutant with residue 284 mutated to Ser, residue 88 mutated to Val, residue 90 mutated to Val, residue 126 mutated to Phe, residue 199 mutated to Val, or residue 379 mutated to Ile, corresponding to SEQ ID NO: 1, and an enzyme for converting rebaudioside A into rebaudioside D, to produce rebaudioside M; preferably, the enzyme for converting stevioside to rebaudioside A is also UGT76G1, UGT76G1 mutant, the enzyme for converting rebaudioside A into rebaudioside D includes: EUGT11, UGT91D2; or the method includes conducting catalyzation with rebaudioside D as a substrate via a glycosyltransferase UGT76G1 mutant with residue 284 mutated to Ser, residue 85 mutated to Val, residue 88 mutated to Val, residue 126 mutated to Phe, residue 199 mutated to Phe, residue 199 mutated to Leu, residue 199 mutated to Val, residue 200 mutated to Ile, residue 203 mutated to Val, residue 379 mutated to Ile, residue 379 mutated to Val, or residue 379 mutated to Trp, corresponding to SEQ ID NO: 1, to produce rebaudioside M; or the method includes conducting catalyzation with steviol as a substrate via a glycosyltransferase UGT76G1 mutant with residue 284 mutated to Ser, residue 88 mutated to Val, residue 90 mutated to Val, residue 126 mutated to Phe, residue 199 mutated to Val, or residue 379 mutated to Ile, corresponding to SEQ ID NO: 1, and an enzyme for converting rebaudioside A or stevioside into rebaudioside D and an enzyme for converting steviol into rebaudioside A or stevioside, to produce rebaudioside M; the enzyme for converting steviol into rebaudioside A or stevioside includes: EUGT11, UGT91D2, UGT74G1, UGT85C2, UGT75L20, UGT75L21, UGT75W2, UGT75T4, UGT85A57, UGT85A58, UGT76G1, UGT76G1 mutant.

In another preferred embodiment, the method also comprises: using an enzyme for recycling UDP glucose; preferably, the enzyme for recycling UDP glucose includes (but is not limited to): AtSUS3.

Another aspect of the invention provides a composition comprising the glycosyltransferase UGT76G1 mutant; or comprising the host cell of any embodiment described above.

Another aspect of the invention provides a kit comprising the glycosyltransferase UGT76G1 mutant of any embodiment described above; or comprising the host cell of any embodiment described above; or the composition described above.

In another preferred embodiment, the composition also includes a pharmaceutically or industrially acceptable carrier.

Other aspects of the disclosure will be apparent to those skilled in the art based on the disclosure herein.

### BRIEF DESCRIPTION OF DRAWINGS

FIG. 1 shows SDS-PAGE of UGT76G1 (53.4 kDa) after Ni NTA purification. Where, P: precipitation; S: Supernatant; F: Flow-through-fluid; W: Washing solution; R: Resin; M: Marker

FIG. 2 shows molecular exclusion purification peak and SDS-PAGE of UGT76G1.

FIG. 3 shows co-crystalline crystals of UGT76G1, steviolbioside and UDP glucose.

FIG. 4 shows chemical structure of rebaudioside B. Circle 1: glycosyl 1; Circle 2: glycosyl 2; Circle 3: glycosyl 3.

FIG. 5 shows binding pocket of rebaudioside B.

FIG. 6 shows gel electrophoresis of mutant PCR products.

FIG. 7 shows mutant protein expression.

FIG. 8 shows H25A and D124N mutants had no catalytic activity on all tested substrates. a. Substrate steviolmonoside; b. Substrate steviolbioside; c. Substrate rubusoside; d. Substrate stevioside; e. Substrate rebaudioside A; f. Substrate rebaudioside D.

FIG. 9 shows effects of the mutant with mutated T284 on different substrates. a. Substrate steviolmonoside; b. Substrate steviolbioside; c. Substrate rubusoside; d. Substrate stevioside; e. Substrate rebaudioside A; f Substrate rebaudioside D.

FIG. 10 shows the catalytic activity of the mutant with mutated 5147 and H155 for substrate steviolmonoside, rubusoside and rebaudioside A is decreased. a. Substrate steviolmonoside; b. Substrate rubusoside; c. Substrate rebaudioside A; d. Substrate stevioside; e. Substrate rebaudioside A; f Substrate rebaudioside D.

FIG. 11 shows T146 and D380 mutations of stable glycosyl 3 affect the catalytic activity of the substrate. The activity of UGT76G1 is 1 and the activities of the mutants relative to 1. a. Substrate steviolmonoside; b. Substrate steviolbioside; c. Substrate rubusoside; d. Substrate stevioside; e. Substrate rebaudioside A; f. Substrate rebaudioside D.

FIG. 12 shows catalytic activity of a double-mutant on substrates rebaudioside A and rebaudioside D. a. Substrate rebaudioside A; b. Substrate rebaudioside D.

FIG. 13 shows production of rebaudioside M by fermentation of recombinant Escherichia coli system.

FIG. 14 shows gel electrophoresis of PCR products when constructing mutants.

FIG. 15 shows SDS-PAGE results of some mutants (l126V, L126F, L379F, L379W, L379V) upon expression and purification.

FIG. 16 shows catalytic activity of mutant on substrate steviolmonoside.

FIG. 17 shows catalytic activity of mutant on substrate steviolbioside.

FIG. 18 shows catalytic activity of mutant on substrate rubusoside.

FIG. 19 shows catalytic activity of mutant on substrate stevioside.

FIG. 20 shows catalytic activity of mutant on substrate rebaudioside A.

FIG. 21 shows catalytic activity of mutant on substrate rebaudioside D.

### DETAILED DESCRIPTION

Upon in depth research, the inventor has revealed a glycosyltransferase UGT76G1 mutant. The catalytic activity, substrate selectivity and/or substrate specificity of the mutant have been changed, which can significantly promote the catalytic activity of 1,3-glycosylation of substrates containing 1,2-diglucosyl, and significantly reduce the catalytic activity of 1,3-glycosylation based on monoglucosyl substrate. When the 1,2-diglucose substrate is rebaudioside D, the glycosyltransferase UGT76G1 mutant of the invention promotes the production of rebaudioside M and reduces the production of by-products. The invention also discloses a series of other mutants that increase or decrease the catalytic activity of glycosyltransferase UGT76G1.

As used herein, unless otherwise specified, the “glycosyltransferase UGT76G1 mutant” and “mutated glycosyltransferase UGT76G1” can be used interchangeably, which refer to the polypeptide after mutation near the substrate binding pocket corresponding to the wild-type glycosyltransferase UGT76G1 or the polypeptide with changed catalytic activity. Preferably, the polypeptide is formed after mutation at position 284, 147, 155, 146, 380, 85, 87, 88, 90, 91, 126, 196, 199, 200, 203, 204 or 379 of the sequence.

The wild-type glycosyltransferase UGT76G1 can be referred as “a protein of amino acid sequence of SEQ ID NO: 1”, or “a functional variant or active fragment of the protein”. Preferably, the wild-type glycosyltransferase UGT76G1 is derived from Stevia rebaudiana. However, it should be understood that the invention also encompasses UGT76G1 homologues from other plants with homology and the same function.

As used herein, “isolated glycosyltransferase UGT76G1” means that the glycosyltransferase UGT76G1 mutant substantially contains no other naturally related proteins, lipids, carbohydrates or other substances. The skilled in the art can purify the glycosyltransferase UGT76G1 mutant by standard protein purification technology. Substantially pure protein can produce a single main band on non-reducing SDS-PAGE.

As used herein, “substrate binding pocket” refers to the position where the glycosyltransferase UGT76G1 interacts (binds) with the substrate in the spatial structure.

The protein of the invention can be a recombinant protein, a natural protein, a synthetic protein, preferably a recombinant protein. The protein of the invention can be a naturally purified product, or a chemically synthesized product, or can be produced by prokaryotic or eukaryotic hosts (such as bacteria, yeast, higher plants, insects and mammalian cells) using recombination technology.

The invention also includes fragments, derivatives and analogues of the glycosyltransferase UGT76G1 mutant. As used herein, the terms “fragments”, “derivatives” and “analogues” refer to proteins that basically maintain the same biological function or activity of the natural glycosyltransferase UGT76G1 mutant of the invention. Functional fragments, derivatives or analogs in the disclosure may be (i) proteins with one or more conservative or non-conservative amino acid substitution (preferably conservative), where the substituted amino acid residues may or may not be one encoded by the genetic code, or (ii) proteins with substituents in one or more amino acid residues, or (iii) proteins formed by having said protein fused with additional amino acid sequence (such as leader sequence or secretory sequence, or sequence used for purification of the protein or proprotein sequence, or fusion protein). In accordance with the teachings provided herein, these fragments, derivatives and analogs are well known to a person skilled in the art. However, the mutation disclosed herein should exist in the amino acid sequence of the glycosyltransferase UGT76G1 mutant and its fragments, derivatives and analogues; preferably, the mutation occurs at amino acid corresponding to residue 284, 147, 155, 146, 380, 85, 87, 88, 90, 91, 126, 196, 199, 200, 203, 204 or 379 of SEQ ID NO: 1.

As used herein, the “glycosyltransferase UGT76G1 mutant” further comprises but is not limited to: deletion, insertion and/or substitution of several (usually 1-20, preferably 1-10, more preferably 1-8, 1-5, 1-3, 1-2) amino acids, and addition or deletion of one or several (usually within 20, preferably within 10, more preferably within 5) amino acids at the C-terminal and/or N-terminal. For example, substitution with amino acids of comparable or similar properties usually does not change protein function in the art. As another example, addition of deletion of one or more amino acids to the C-terminus and/or N-terminus usually does not change the function of a protein either. The term also includes the active fragments and active derivatives of the glycosyltransferase UGT76G1 mutant. However, these variants should comprise the mutation described herein; preferably, the mutation occurs at amino acid corresponding to residue 284, 147, 155, 146, 380, 85, 87, 88, 90, 91, 126, 196, 199, 200, 203, 204 or 379 of SEQ ID NO: 1. In the present invention, the term “glycosyltransferase UGT76G1 mutant” also includes (but is not limited to): a derived protein having more than 80%, more preferably more than 85%, more preferably more than 90%, and further more preferably more than 95%, such as more than 98% and more than 99% sequence identity with the amino acid sequence of the glycosyltransferase UGT76G1 mutant and retaining the activity of the mutant. Similarly, these derived proteins should comprise the mutation described herein; preferably, the mutation occurs at amino acid corresponding to residue 284, 147, 155, 146, 380, 85, 87, 88, 90, 91, 126, 196, 199, 200, 203, 204 or 379 of SEQ ID NO: 1.

The invention also provides a polynucleotide sequence encoding a glycosyltransferase UGT76G1 mutant of the invention or a conservative variant protein thereof.

The polynucleotide sequences herein can be in the form of DNA or RNA. Forms of DNA include cDNA, genomic DNA or artificially synthesized DNA. DNA can be single-stranded or double-stranded. The DNA may be coding strand or non-coding strand.

The polynucleotide encoding the mature protein of the mutant disclosed herein includes: the coding sequence only encoding the mature protein; the coding sequence encoding the mature protein and a various additional coding sequence; the coding sequence encoding the mature protein (and an optional additional coding sequence) and a noncoding sequence.

The term “polynucleotide encoding a/the protein” can include a polynucleotide encoding the protein, or a polynucleotide that further includes additional coding and/or non-coding sequences.

The disclosure also relates to vectors comprising the polynucleotide of the disclosure, as well as host cells genetically engineered using the vectors or coding sequences of the glycosyltransferase UGT76G1 mutant disclosed herein, and a method for producing the protein of the invention by recombination technology.

Through conventional recombinant DNA technology, the polynucleotide sequence of the invention can be used to express or produce the recombinant glycosyltransferase UGT76G1 mutant. Generally, there are the following steps:

(1) Transforming or transducing a suitable host cell with a polynucleotide (or variant) encoding a glycosyltransferase UGT76G1 mutant of the present invention, or with a recombinant expression vector containing the polynucleotide;

(2) culturing the host cell in a suitable medium;

(3) isolating and purifying proteins from the medium or cell.

In the invention, the polynucleotide sequence of glycosyltransferase UGT76G1 mutant can be inserted into the recombinant expression vector. The term “recombinant expression vector” refers to bacterial plasmid, phage, yeast plasmid, plant cell virus, mammalian cell virus or other vectors well known in the art. In short, any plasmid or vector can be used, provided that it can replicate and be stable in the host. An important characteristic of an expression vector is that it usually contains an origin of replication, a promoter, a marker gene and a translation control element.

Suitable methods for constructing expression vector which comprises the coding DNA sequence of the glycosyltransferase UGT76G1 mutant and appropriate transcriptional/translational control signals are well known to the person skilled in the art. These methods include in vitro recombinant DNA technology, DNA synthesis technology, in vivo recombinant technology and so on. Said DNA sequence may be effectively linked to a proper promoter in the expression vector to direct mRNA synthesis. Expression vector further comprises a ribosome binding site for the imitation of translation, and a transcription terminator. The expression vector preferably contains one or more selective marker genes to provide phenotypic traits for the selection of transformed host cells.

Vectors containing the above appropriate DNA sequences and appropriate promoters or regulatory sequences can be used to transform appropriate host cells so that they can express proteins.

In this disclosure, the host cells can be prokaryotic cells, such as bacterial cells; or lower eukaryotic cells, such as yeast cells; or higher eukaryotic cells, such as plant cells. Examples include Escherichia coli, Bacillus subtilis, Streptomyces, Agrobacterium; eukaryotic cells, such as yeast, plant cells, etc. In a specific embodiment of the invention, Escherichia coli is used as the host cells.

The choice of appropriate carrier, promoter, enhancer and host cells is evident to a person of ordinary skills in the art.

In the invention, the substrate containing 1,2-diglucoside includes but is not limited to steviolbioside, stevioside, rebaudioside D or rebaudioside E. The monoglucosyl substrate includes but is not limited to steviolmonoside, rubusoside, rebaudioside A, steviol 19-O-glucose ester and kaurenic acid 19-O-glucose ester.

Based on the information of the mutant glycosyltransferase UGT76G1 described herein, those skilled in the art know how to use the mutant to perform 1,3-glycosylation of the substrate containing 1,2-diglucosyl.

For example, the product of 1,3-glycosylation is rebaudioside M. Rebaudioside D is catalyzed by the glycosyltransferase UGT76G1 mutant to obtain rebaudioside M. Various intracellular or extracellular preparation methods are included in the invention or can be applied to the invention.

Considering the cost of the substrate, in a preferred embodiment of the disclosure, the method includes conducting catalyzation with rebaudioside A as substrate via a glycosyltransferase UGT76G1 mutant with residue 284 mutated to Ser, residue 85 mutated to Val, residue 126 mutated to Phe, residue 199 mutated to Phe, residue 199 mutated to Leu or residue 203 mutated to Val, corresponding to SEQ ID NO: 1, and an “enzyme for converting rebaudioside A into rebaudioside D”, to produce rebaudioside M. Since the preparation of rebaudioside M and its upstream reactions are known in the art, those skilled in the art understand what the “enzyme for converting rebaudioside A into rebaudioside D” is in the art. Preferably, the “enzyme for converting rebaudioside A into rebaudioside D” can be EUGT11, UGT91D2 (SEQ ID No: 5).

In another preferred embodiment, the method includes conducting catalyzation with stevioside as a substrate via an “enzyme for converting stevioside to rebaudioside A”, a glycosyltransferase UGT76G1 mutant with residue 284 mutated to Ser, residue 88 mutated to Val, residue 90 mutated to Val, residue 126 mutated to Phe, residue 199 mutated to Val, or residue 379 mutated to Ile, corresponding to SEQ ID NO: 1, and an “enzyme for converting rebaudioside A into rebaudioside D”, to produce rebaudioside M. Similarly, based on the knowledge in the art, those skilled in the art understand what the “enzyme for converting stevioside to rebaudioside A” is in the art. Preferably, the “enzyme for converting stevioside to rebaudioside A” is also UGT76G1, UGT76G1 mutant; and the “enzyme for converting rebaudioside A into rebaudioside D” can be EUGT11, UGT91D2 (SEQ ID NO: 5).

In another preferred embodiment, the method includes conducting catalyzation with rebaudioside D as a substrate via a glycosyltransferase UGT76G1 mutant with residue 284 mutated to Ser, residue 85 mutated to Val, residue 88 mutated to Val, residue 126 mutated to Phe, residue 199 mutated to Phe, residue 199 mutated to Leu, residue 199 mutated to Val, residue 200 mutated to Ile, residue 203 mutated to Val, residue 379 mutated to Ile, residue 379 mutated to Val, or residue 379 mutated to Trp, corresponding to SEQ ID NO: 1, to produce rebaudioside M.

In another preferred embodiment, the method includes conducting catalyzation with steviol as a substrate via a glycosyltransferase UGT76G1 mutant with residue 284 mutated to Ser, residue 88 mutated to Val, residue 90 mutated to Val, residue 126 mutated to Phe, residue 199 mutated to Val, or residue 379 mutated to Ile, corresponding to SEQ ID NO: 1, and an “enzyme for converting rebaudioside A or stevioside into rebaudioside D” and an “enzyme for converting steviol into rebaudioside A or stevioside”, to produce rebaudioside M. Similarly, based on the knowledge in the art, those skilled in the art understand what the “enzyme for converting steviol into stevioside or rebaudioside A” is in the art. Preferably, the “enzyme for converting steviol into stevioside or rebaudioside A” include (but are not limited to): EUGT11, UGT91D2, UGT74G1, UGT85C2, UGT75L20, UGT75L21, UGT75W2, UGT75T4, UGT85A57, UGT85A58.

The above method for preparing rebaudioside M can be carried out in or out of cells. As a preferred embodiment of the disclosure, a method for producing rebaudioside M in cells is provided: transforming into host cells the coding genes of the glycosyltransferase UGT76G1 mutant having amino acid sequence corresponds SEQ ID NO: 1 and residue 284 mutated to Ser, together with the above “enzyme for converting rebaudioside A into rebaudioside D”, “enzyme for converting stevioside into rebaudioside A”, “enzyme for catalyzing steviol to stevioside or rebaudioside A” and/or “enzyme converting rebaudioside A or stevioside to rebaudioside D”, culturing the cells to produce rebaudioside M.

The disclosure also provides a series of mutants of glycosyltransferase UGT76G1 having decreased catalytic activity, wherein mutations occur at residue 147, 155, 146 or 380 corresponding to SEQ ID NO: 1. For example, they can be used in a production system in which rebaudioside M is not the end product, thereby reducing the amount of substrate converted to rebaudioside M and accumulate intermediate products. The decrease catalytic activity of glycosyltransferase UGT76G1 is conducive to the controlling of generating different products and is meaningful for the production of different products.

Compared with the prior art, the progress of the disclosure is that the glycosyltransferase UGT76G1 mutant obtained by the disclosure efficiently and specifically catalyzes the glycosylation of the 3′ glucose group in the structure of stevia glycoside in vitro. As compared with the wild-type protein, the mutant catalyzes rebaudioside D into rebaudioside M much more efficiently, and the by-product rebaudioside I formed by rebaudioside A is greatly reduced.

The disclosure is further illustrated by the specific examples described below. It should be understood that these examples are merely illustrative, and do not limit the scope of the present disclosure. The experimental methods without specifying the specific conditions in the following examples generally used the conventional conditions, such as those described in J. Sambrook, Molecular Cloning: A Laboratory Manual (3rd ed. Science Press, 2002) or followed the manufacturer's recommendation.

Materials and Instruments

PCR primers were synthesized by Shanghai Sangon Biotech Co., Ltd. or Genscript Biotechnology Co., Ltd. Sanger sequencing was entrusted to Shanghai Sangon Biotech Co., Ltd. PCR gel recovery kit, and plasmid extraction kit were available from Axygen; PCR high fidelity enzyme PrimeSTAR Max DNA Polymerase is available from Takara; restriction endonuclease and T4 ligase are available from New England Biolabs (NEB). Seamless cloning kit was purchased from Vazyme Biotechnology Co., Ltd. E. coli DH10B was used for cloning construction, BL21(DE3) was used for protein expression. Vector pETDuet-1 was used for gene cloning and protein expression. Wild-type UGT76G1 and EUGT11 were synthesized by Genscript Biotechnology Co., Ltd. and optimized with E. coli codon. Ni NTA was purchased from Qiagen. Superdex 200 column (GE Healthcare) was used for protein molecular exclusion and purification. Molecular diamond (Hampton research, America) was used to screen protein crystallization condition.

Standard compounds steviol, rebaudioside A, stevioside and steviolbioside were purchased from Shanghai Yuanye Biotechnology Co., Ltd., rubusoside was purchased from Nanjing Guangrun Biological Products Co., Ltd., rebaudioside D and rebaudioside M were purchased from Sichuan YingjiaHesheng Technology Co., Ltd. UDP glucose was purchased from Beijing Zhongtai biological Co., Ltd. Other reagents are analytical grade reagent or chromatographic grade reagent, purchased from Sinopharm Chemical Reagent Co., Ltd. IPTG, MgCl₂, PMSF and ampicillin were purchased from Sangon Biotech (Shanghai) Co., Ltd. DNase I (10 mg/ml) was purchased from Shanghai yanye biotechnology service center. PMSF was purchased from Sigma China.

PCR was conducted on Arktik Thermal Cycler (Thermo Fisher Scientific); ZXGP-A2050 Incubator (Zhicheng) and ZWY-211G Constant Temperature Oscillator (Zhicheng) were used for culture; high-speed freezing Centrifuge 5418R and Centrifuge 5418 (Eppendorf) were used for centrifugation. Vacuum concentration was performed with Concentrator Plus (Eppendorf); OD₆₀₀ was detected using UV-1200 Ultraviolet/Visible Spectrophotometer (Shanghai Mapada Instrument Co., Ltd.). Rotary evaporation system consists of IKA RV 10 Digital Rotary Evaporator (IKA), MZ 2C NT Chemical Diaphragm Pump and CVC3000 vacuum controller (Vacuubrand). C3 high pressure cell crusher (Sunnybay Biotech Co., Canada) was used for cell broken. Dionex UltiMate 3000 Liquid Chromatography System (Thermo Fisher Scientific) was used for HPLC. The crystal diffraction data were collected at Shanghai Synchrotron Radiation Facility BL19U and analyzed by HKL3000 package for structure.

### Example 1. Expression, Purification, Crystallization and Structure Analysis of UGT76G1 Protein

1. Construction of Wild-Type UGT76G1 Expression Vector pQZ11

The target gene was amplified with specific primer pairs (Table 1) and with the codon-optimized UGT76G1 gene cloning vector as the template. The PCR product was cloned into BamHI/HindIII of vector pETDuet1, and the obtained expression vector pQZ11 was verified by sequencing.

TABLE 1
Primers used in the construction of
wild-type UGT76G1 expression vector

| Primer Name | Sequence |
|---|---|
| Primer_F | ATTCTGGATCCATGGAAAACAAAAC |
| | (SEQ ID NO: 94) |
| Primer_R | CGCAAGCTTTTAACTTTACAGAGAA |
| | (SEQ ID NO: 95) |

2. Protein Expression and Purification

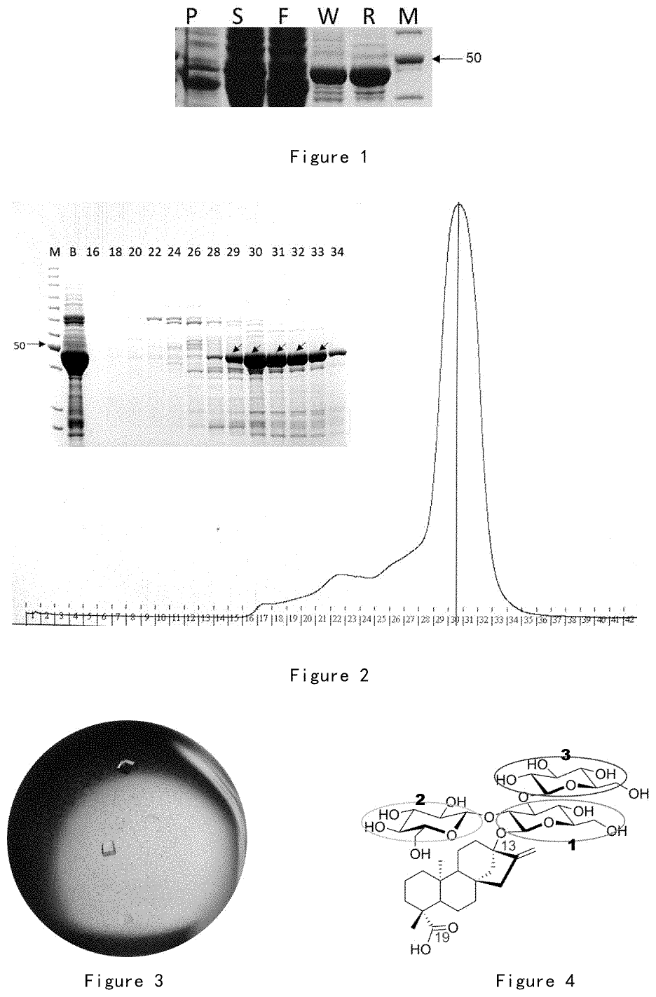

E. coli BL21 (DE3) harboring wild-type UGT76G1 expression vector pQZ11 cultured overnight was transferred to 1 L LB at 1% (v/v) and cultured at 37° C. and 200 rpm until OD₆₀₀≈1.0. The final concentration of 0.1 mM IPTG was used for induction, and the cells were collected after 18 hours of overnight culture at 16° C. Resuspending the cells with resuspension buffer, adding 1 mM PMSF, 2 mM MgCl₂, and 5 μg/mL DNase I and mixing well, and then holding on ice for 30 min. After the cells were lysed using a high-pressure cell crusher and centrifuged at high speed, the supernatant was spin-incubated with Ni-NTA purification resin (4° C.), and then eluted with 6-10 column volumes of 25 mM imidazole. Finally, 10 column volumes of 250 mM imidazole were used to elute the purified resin (FIG. 1), and the solution was concentrated to 20 mg/mL before size exclusion purification. The protein at the peak of FPLC was collecting and used to screen crystals after verification by SDS-PAGE (FIG. 2).

SrUGT76G1_wide-type (SEQ ID NO: 1):
MENKTETTVRRRRRIILFPVPFQGHINPILQLANVLYSKGFSITIFHTNFNKPKTSNYPHFTFRFILDN
DPQDERISNLPTHGPLAGMRIPIINEHGADELRRELELLMLASEEDEEVSCLITDALWYFAQSVADSLNLR
RLVLMTSSLFNFHAHVSLPQFDELGYLDPDDKTRLEEQASGFPMLKVKDIKSAYSNWQILKEILGKMIK
QTKASSGVIWNSFKELEESELETVIREIPAPSFLIPLPKHLTASSSSLLDHDRTVFQWLDQQPPSSVLYVSF

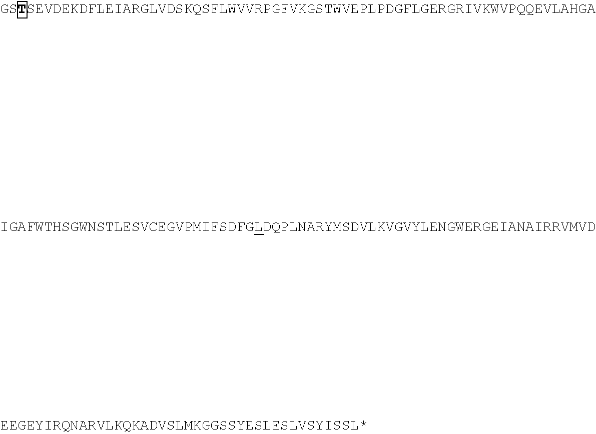

3. Protein Crystallization and Structure Analysis

According to the chromatographic results of molecular exclusion purification of UGT76G1 and SDS-PAGE results, the concentration of the protein with the highest purity was determined and concentrated to 5 mg/mL and 10 mg/mL, respectively. Adding the small molecule substrate according to the molar ratio of concentrated protein to substrate concentration of 1:20, and generating high-quality crystals of complex of UGT76G1 and substrate (steviolbioside, UDP-glucose) by sitting drop at 20° C. (FIG. 3), with the resolution up to 2.5 Å.

By analyzing the structure of UGT76G1 based on the diffraction data, the inventors obtained the complex structure of UGT76G1, rebaudioside B (the catalyzing product of UGT76G1), and UDP.

### Example 2. Construction and Expression of Mutant Protein

According to the complex structure of UGT76G1-substrate rebaudioside B (FIG. 4) and UDP and based on repeated verifications, the inventor located the substrate binding pocket and identified several key amino acids in the substrate binding pocket (FIG. 5), which interact with glycosyl donor, glycosyl acceptor or aglycon core, respectively. Amino acids were divided into 4 categories according to their functions in the glycosylation process (Table 2). These amino acids were subjected to single-point or multiple-point mutations. Through in vitro enzymatic tests, the mutant proteins were analyzed for catalytic activity and substrate recognition specificity changes in the glycosylation process.

TABLE 2
Amino acid mutation

| Amino acid | Function | Mutation |
|---|---|---|
| H25 | Catalyzing | A |
| D124 | Catalyzing | N |
| T284 | Stabilization of glycosyl 1 | A/S |
| S147 | Stabilization of glycosyl 2 | A/N/Q |
| H155 | Stabilization of glycosyl 2 | A/Y |
| T146 | Stabilization of glycosyl 3 | A/N/S |
| D380 | Stabilization of glycosyl 3/ | E/N/S/T |
| | glycosyl donor recognition | |

1. Mutant Construction

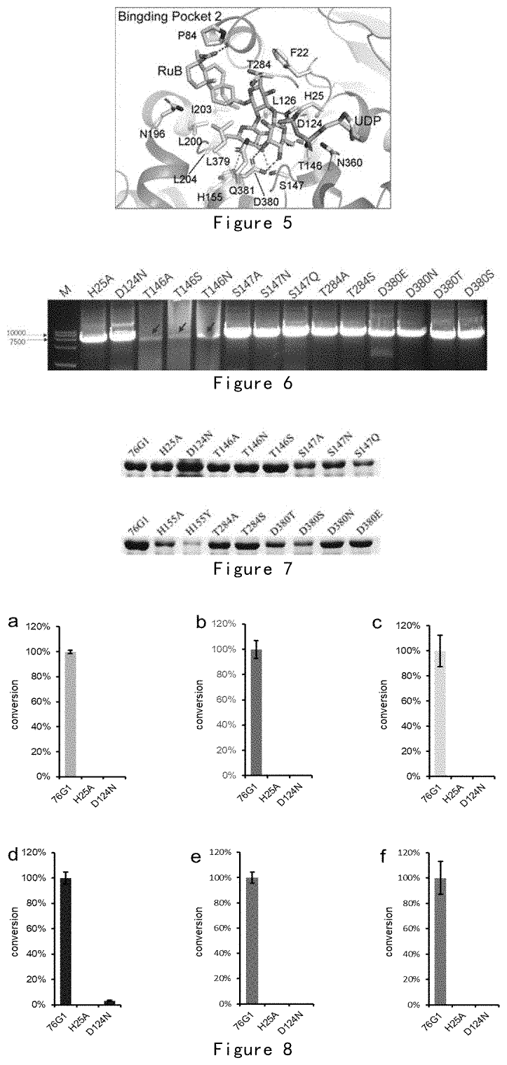

The genes of mutants were amplified by PCR (FIG. 6), with primers containing point mutation (Table 3) and with wild-type UGT76G1 expression vector pQZ11 as a template, and was transformed into DH10B, and verified by sequencing.

TABLE 3
Primers used to amplify mutants

| Primer Name | Sequence (5′→3′) |
|---|---|
| Primer_D124N_F | AAGTTTCTTGCCTGATCACCAACGCGCTGTGGT (SEQ ID NO: 6) |
| Primer_D124N_R | GTTGGTGATCAGGCAAGAAACTTCTTCGTCTTC (SEQ ID NO: 7) |
| Primer_D380E_F | TCTTCTCTGACTTCGGTCTGGAACAGCCGCTGA (SEQ ID NO: 8) |
| Primer_D380E_R | TTCCAGACCGAAGTCAGAGAAGATCATCGGAAC (SEQ ID NO: 9) |
| Primer_D380N_F | TCTTCTCTGACTTCGGTCTGAACCAGCCGCTGA (SEQ ID NO: 10) |
| Primer_D380N_R | GTTCAGACCGAAGTCAGAGAAGATCATCGGAAC (SEQ ID NO: 11) |
| Primer_D380S_F | TCTTCTCTGACTTCGGTCTGTCTCAGCCGCTGA (SEQ ID NO: 12) |
| Primer_D380S_R | AGACAGACCGAAGTCAGAGAAGATCATCGGAAC (SEQ ID NO: 13) |
| Primer_D380T_F | TCTTCTCTGACTTCGGTCTGACCCAGCCGCTGA (SEQ ID NO: 14) |
| Primer_D380T_R | GGTCAGACCGAAGTCAGAGAAGATCATCGGAAC (SEQ ID NO: 15) |
| Primer_H25A_F | TCCCGGTTCCGTTCCAGGGTGCGATCAACCCGA (SEQ ID NO: 16) |
| Primer_H25A_R | CGCACCCTGGAACGGAACCGGGAACAGGATGAT (SEQ ID NO: 17) |
| Primer_S147A_F | GTCGTCTGGTTCTGATGACCGCGTCTCTGTTCA (SEQ ID NO: 18) |
| Primer_S147A_R | CGCGGTCATCAGAACCAGACGACGCAGGTTCAG (SEQ ID NO: 19) |
| Primer_S147N_F | GTCGTCTGGTTCTGATGACCAACTCTCTGTTCA (SEQ ID NO: 20) |
| Primer_S147N_R | GTTGGTCATCAGAACCAGACGACGCAGGTTCAG (SEQ ID NO: 21) |
| Primer_S147Q_F | GTCGTCTGGTTCTGATGACCCAGTCTCTGTTCA (SEQ ID NO: 22) |
| Primer_S147Q_R | CTGGGTCATCAGAACCAGACGACGCAGGTTCAG (SEQ ID NO: 23) |
| Primer_T146A_F | TGCGTCGTCTGGTTCTGATGGCGTCTTCTCTGT (SEQ ID NO: 24) |
| Primer_T146A_R | CGCCATCAGAACCAGACGACGCAGGTTCAGAGA (SEQ ID NO: 25) |
| Primer_T146N_F | TGCGTCGTCTGGTTCTGATGAACTCTTCTCTGT (SEQ ID NO: 26) |
| Primer_T146N_R | GTTCATCAGAACCAGACGACGCAGGTTCAGAGA (SEQ ID NO: 27) |
| Primer_T146S_F | TGCGTCGTCTGGTTCTGATGTCTTCTTCTCTGT (SEQ ID NO: 28) |
| Primer_T146S_R | AGACATCAGAACCAGACGACGCAGGTTCAGAGA (SEQ ID NO: 29) |
| Primer_T284A_F | TGTACGTTTCTTTCGGTTCTGCGTCTGAAGTTG (SEQ ID NO: 30) |
| Primer_T284A_R | CGCAGAACCGAAAGAAACGTACAGAACAGAAGA (SEQ ID NO: 31) |
| Primer J2845_F | TGTACGTTTCTTTCGGTTCTTCTTCTGAAGTTG (SEQ ID NO: 32) |
| Primer_T284S_R | AGAAGAACCGAAAGAAACGTACAGAACAGAAGA (SEQ ID NO: 33) |
| 76G1H155A F | TTCAACTTCCACGCGGCGGTTTCTCTGC (SEQ ID NO: 34) |
| 76G1H155A R | CGCCGCGTGGAAGTTGAACAG (SEQ ID NO: 35) |
| 76G1H155Y F | TTCAACTTCCACGCGTATGTTTCTCTGC (SEQ ID NO: 36) |
| 76G1H155Y R | ATACGCGTGGAAGTTGAACAG (SEQ ID NO: 37) |

2. Expression and Purification of Mutant Protein

The expression vector containing the mutant that was verified to be correct was transformed into E. coli expression host BL21(DE3). BL21 (DE3) harboring mutant expression vector was cultured overnight and transferred to 1 L LB at 1% (v/v) and cultured at 37° C. and 200 rpm until OD600 about 1.0. The final concentration of 0.1 mM IPTG was used for induction, and the cells were collected after 18 hours of overnight culture at 16° C. The preparation process of crude enzyme is the same as that of wild-type UGT76G1. The crude enzyme solution was spin-incubated with 1 mL Ni-NTA purification resin (4° C.), and then eluted with 6-10 column volumes of 25 mM imidazole. Finally, 1 mL of 250 mM imidazole was used to incubate at 4° C. for 10-30 minutes to elute the target protein. The BSA method was used to determine the concentration of the target protein, which was stored in 50% glycerol (−20° C.). As shown in FIG. 7, all mutant proteins were expressed. The mutant proteins were used for in vitro enzyme activity testing later.

### Example 3. Functional Verification of Mutant Protein In Vitro

1. Enzymic Reaction of Mutant In Vitro

The enzymic reaction system includes: 10 μg protein, 1.5 mM UDP-glucose, 250 μM glycosyl acceptor substrate buffer (20 mM Tris-HCl, pH=8.0, 100 mM NaCl). The reaction of each mutant protein for the same substrate was repeated three times.

Reaction conditions: 37° C., 30 min. The reaction was quenched with an equal volume of methanol. After vigorous shaking, the reaction was centrifuged at 12000 rpm for 30 min. The supernatant was used for HPLC detection. Detection method: mobile phase A (acetonitrile)-mobile phase B (water) gradient elution. The peak area of the catalytic product of the mutant was calculated and compared with the peak area of the catalytic product of wild-type UGT76G1.

2. Catalytic Activity and Substrate Specificity of Mutant

1) The results of in vitro functional verification are shown in FIG. 8. H25/D124 directly participates in the deprotonation of glycosylation site, and H25A and D124N mutants lose catalytic activity on all substrates.

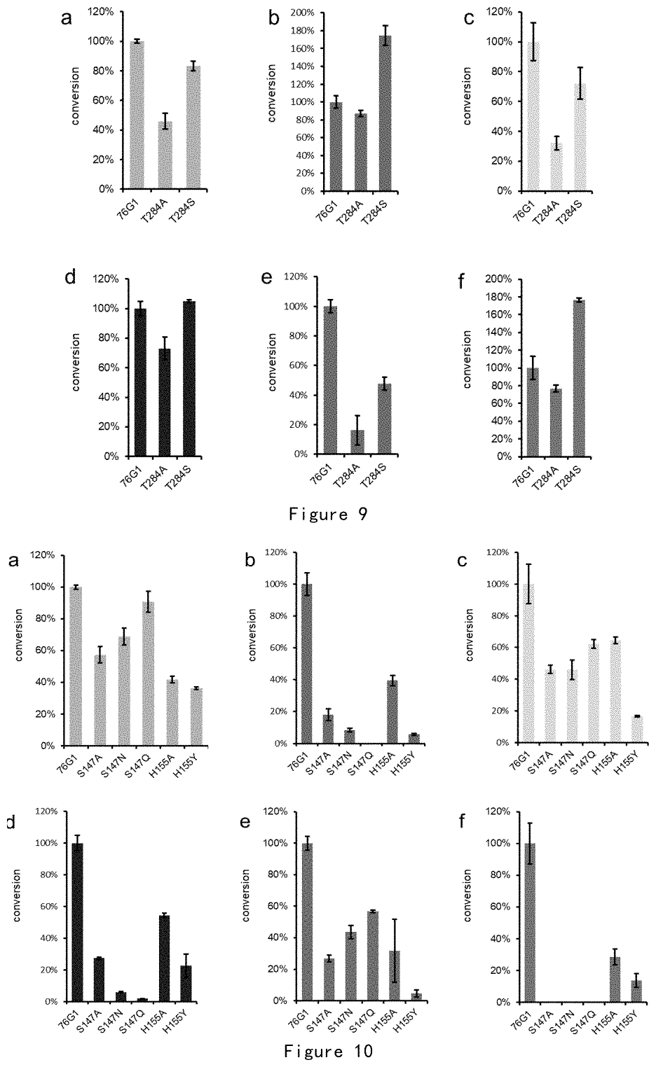

2) The T284 site stabilizes the first glycosyl in the substrate structure. After T is mutated to A, the catalytic activity of the enzyme on all substrates is reduced, and the mutation to S can significantly change the catalytic activity of the enzyme on the substrate (FIG. 9). The relative activity of mutant T284S on the substrates steviolbioside, stevioside and rebaudioside D increased by 74.6%, 4.9%, 76.5%, respectively, and the activity on the substrate steviolmonoside, rubusoside, rebaudioside A decreased by 16.7%, 27.9%, and 52.4%, respectively. The inventors analyzed the substrate structure and found that the three substrates with increased relative catalytic activity have sophorosyl (1,2-diglucosyl), on which 1,3-glycosylation is carried out. Meanwhile, the relative catalytic activity of substrates that directly undergo 1,3-glycosylation based on monoglucosyl substrate is decreased.

3) S147 and H155 stabilize the second glycosyl in the substrate structure. Mutants S147A, S147N, S147Q, H155A, and H155Y had reduced relative catalytic activity on all tested substrates (FIG. 10). It shows that S147 and H155 mutations not only destroy the stability of the second glycosyl, but also affect the binding of the substrate molecule and the enzyme.

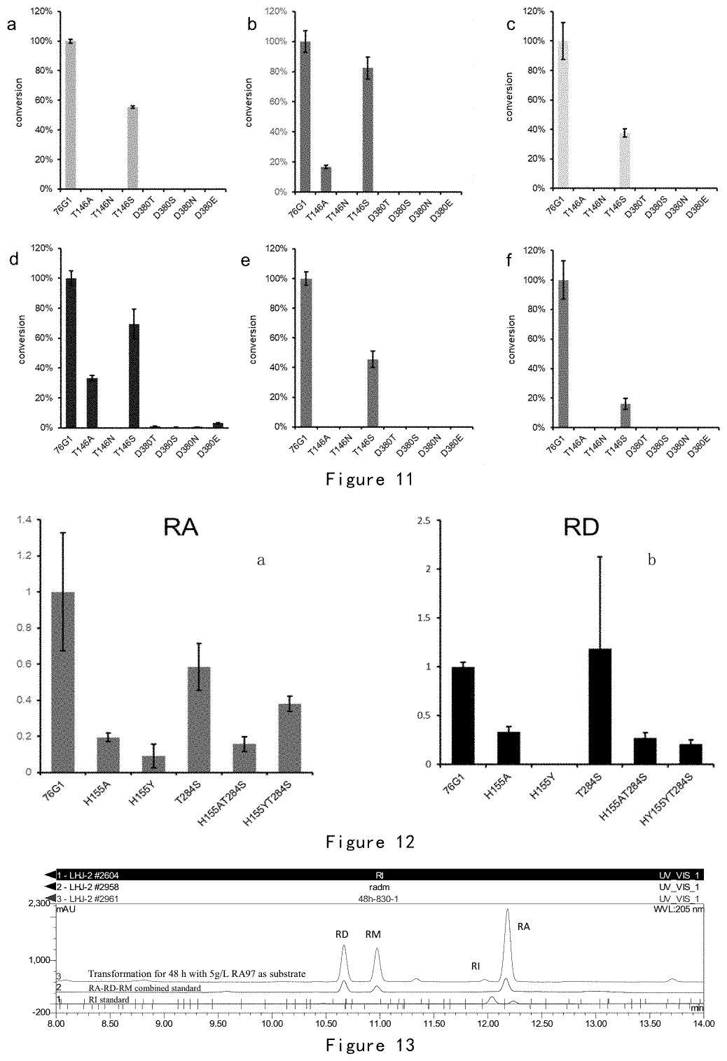

4) The T146A, T146N, and T146S mutants that stabilize the third glycosyl group have reduced catalytic activity on the test substrate, while the D380T, D380S, D380N, and D380E mutants completely lose activity on the substrate (FIG. 11). According to the protein-substrate crystal structure, in addition to interacting with the third glycosyl group of the catalysate, D380 also interacts with the glycosyl donor substrate through hydrogen bonds. Therefore, the mutation D380 may affect the recognition of glycosyl donors, so that the activity of the enzyme on the substrate is completely lost.

### Example 4. Fermentation and Production of Rebaudioside M Using a Recombinant E. coli System Containing Mutants

As a new generation of natural sweetener, rebaudioside M has a better taste than the main stevioside and rebaudioside A in the market. At present, stevioside and rebaudioside A can be obtained cheaply by extracting from natural plants, while rebaudioside M is expensive because of its scarce content in plants. The inventors introduced the two glycosyltransferase genes EUGT11 and UGT76G1 required to synthesize rebaudioside M into the recombinant E. coli system, and converted stevioside and rebaudioside A into high-value products, rebaudioside M, through enzymatic synthesis. Due to the heterogeneity of the substrate of UGT76G1, it can also convert the substrate rebaudioside A into the by-product rebaudioside I. Therefore, the inventors considered the mutant T284S (SEQ ID NO: 2) of UGT76G1, which not only has higher catalytic activity for converting rebaudioside D to the target product rebaudioside M, but also has reduced activity for the substrate rebaudioside A, thereby decreasiong the proportion of by-products.

>SrUGT76G1_T284S
(SEQ ID NO: 2)
MENKTETTVRRRRRIILFPVPFQGHINPILQLANVLYSKGFSITIFHTNFNKPKTSNYPHFTFRFILDN
DPQDERISNLPTHGPLAGMRIPIINEHGADELRRELELLMLASEEDEEVSCLITDALWYFAQSVADSLNLR
RLVLMTSSLFNFHAHVSLPQFDELGYLDPDDKTRLEEQASGFPMLKVKDIKSAYSNWQILKEILGKMIK
QTKASSGVIWNSFKELEESELETVIREIPAPSFLIPLPKHLTASSSSLLDHDRTVFQWLDQQPPSSVLYVSF

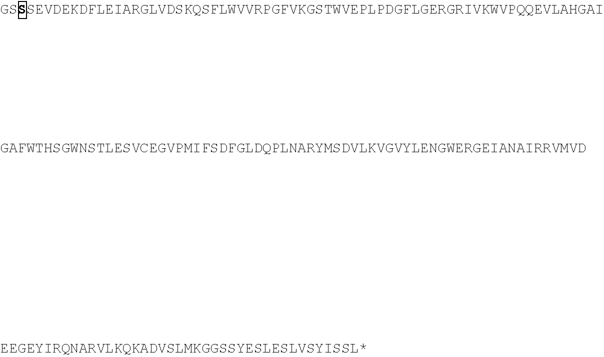

1. Plasmid Construction

EUGT11 gene (encoding protein containing the amino acid sequence of SEQ ID NO: 3) was amplified by PCR with EUGT11 (codon optimized) cloning vector as templates. AtSUS3 gene (encoding sucrose synthase 3 (SEQ ID NO: 4), used for recycling of UDP-glucose) was amplified by PCR with Arabidopsis cDNA as templates. The EUGT11 gene and AtSUS3 gene were introduced between the BamHI/HindIII site and the FseI/KpnI site of pDuet-1, respectively, to form the plasmid pLW108. The mutant gene was amplified by PCR using the mutant UGT76G1 T284S expression vector as a template, and with primers designed to add the homology arms based on the template. UGT76G1 T284s gene was introduced into pLW108 at the downstream of AtSUS3 gene by seamless cloning to form plasmid pHJ830. The plasmid was used to simultaneously express EUGT11, AtSUS3 and UGT76G1 T284S.

TABLE 3
Primers used to construct plasmid

| Primer Name | Sequence(5′→3′) |
|---|---|
| YF09_F | CGC GGATCCATGGACTCCGGCTACTCCTCC (SEQ ID NO: 38) |
| YF09_R | AAGCTT TCAATCCTTGTAAGATCTCAATTGC (SEQ ID NO: 39) |
| Ats3InfuYF09- | CTCAATTGGATATCGGCCGGCCATGGCAAACCCTAAG (SEQ ID NO: |
| Fse | 40) |
| Ats3InfuYF09- | TTTACCAGACTCGAGGGTACCTCAGTCATCGGCGGT (SEQ ID NO: |
| Kpn | 41) |
| 830VF | CTCGAGTCTGGTAAAGAAAC (SEQ ID NO: 42) |
| 830VR | ATTGGTACCTCAGTCATCGGCGG (SEQ ID NO: 43) |
| 830inF | CCGATGACTGAGGTACCAATAATTTTGTTTAACTTTAAG (SEQ ID |
| | NO: 44) |
| 830inR | GTTTCTTTACCAGACTCGAGTTACAGAGAAGAGATGTAAG (SEQ ID |
| | NO: 45) |

2. Production of Rebaudioside M by Fermentation of Recombinant Escherichia coli System.

The above plasmids were transformed into E. coli BL21. Monoclonal colony was selected and inoculated in 10 ml LB medium (Amp=100 μg/mL), cultured at 37° C. for 4 hours. Then the mixture was inoculated in 1 L LB medium at 1%, cultured at 37° C. for 2 hours until OD600=0.5. The culture was cooled to 22° C., added with IPTG (final concentration 100 μM), and inducted for 20 h. The bacteria were concentrated and collected for resting cell transformation reaction. The reaction system is shown in Table 4. After 48 h, the samples were collected for HPLC detection.

The fermentation results showed (FIG. 13) that within 48 hours, about 50% of rebaudioside A (RA) was converted into rebaudioside D (RD) (25%) and rebaudioside M (RM) (25%), and the proportion of by-product rebaudioside I (RI) was less than 1%.

TABLE 4
Resting cell transformation reaction system

| Component | Dosage (for 1 L) |
|---|---|
| Bacteria | OD₆₀₀ = 100 |
| Sodium phosphate buffer | 100 mM, pH 8.0 |
| Trisodium citrate | 23.5 g (60 mM) |
| Sucrose | 400 g (40%, W/V) |
| ZnCl₂ | 0.1363 g (1 mM) |
| Rebaudioside A | 5 g/L (≈5 mM) |

### Example 5. Functional Verification of Diterpene Core Related Mutant In Vitro

1. Mutant Construction

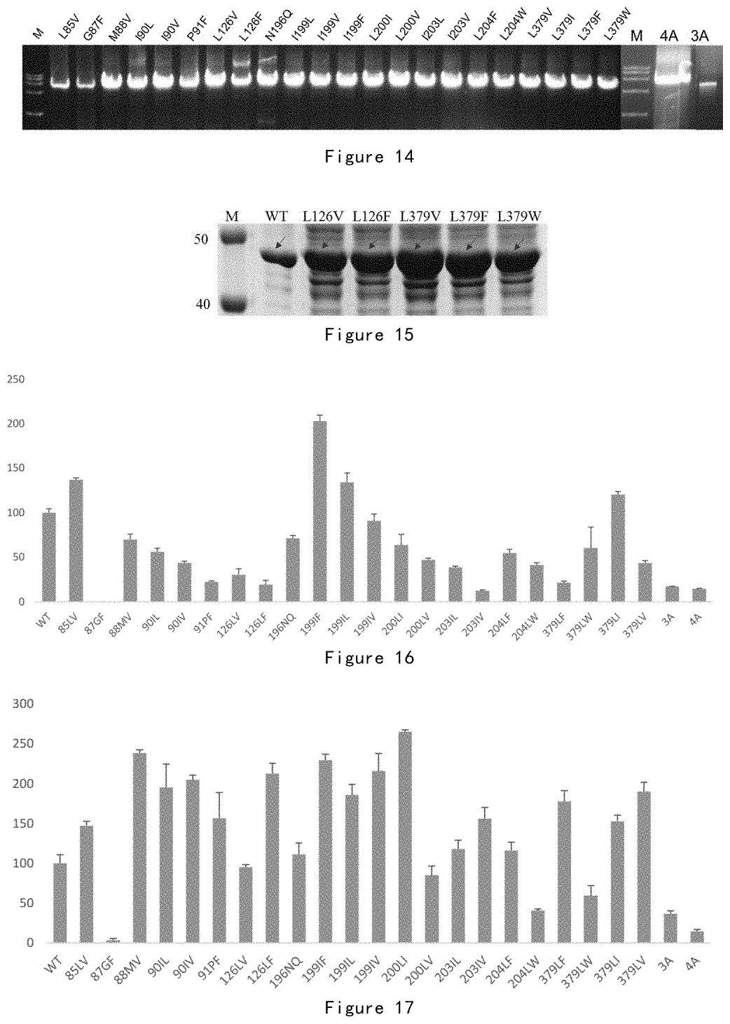

Point mutation was performed on wild-type SrUGT76G1, including residues 85, 87, 88, 90, 91, 126, 196, 199, 200, 203, 204 and 379. The primers for point mutation were shown in Table 5. PCR cloning was performed with the expression vector pQZ11 containing wild-type SrUGT76G1 as a template. Mutant 3A refers to the combined mutant having residues 199, 200 and 203 mutated, and mutant 4A refers to the combined mutant having residues 199, 200, 203 and 204 mutated. The gel electrophoresis results of PCR products in FIG. 14 showed that 24 mutations were successfully amplified. After Dpn I digestion, genes were transformed into E. coli DH10B and verified by sequencing.

TABLE 5
PCR primers

| Primer Name | Sequence |
|---|---|
| Primer_L85V_F | CCGACCCACGGTCCGGTTGCGGGTATGCGTATC (SEQ ID NO: 46) |
| Primer_L85V_R | CGGACCGTGGGTCGGCAGGTTAGAGATACG (SEQ ID NO: 47) |
| Primer_G87F_F | GGTCCGCTGGCGTTCATGCGTATCCCGATC (SEQ ID NO: 48) |
| Primer_G87F_R | GAACGCCAGCGGACCGTGGGTCGG (SEQ ID NO: 49) |
| Primer_M88V_F | GGTCCGCTGGCGGGTGTTCGTATCCCGATC (SEQ ID NO: 50) |
| Primer_M88V_R | ACCCGCCAGCGGACCGTGGGTC (SEQ ID NO: 51) |
| Primer_I90L_F | CTGGCGGGTATGCGTCTGCCGATCATCAACGAAC (SEQ ID NO: 52) |
| Primer_I90L_R | ACGCATACCCGCCAGCGGACCGTG (SEQ ID NO: 53) |
| Primer_I90V_F | CTGGCGGGTATGCGTGTTCCGATCATCAACGAAC (SEQ ID NO: 54) |
| Primer_I90V_R | ACGCATACCCGCCAGCGGACCG (SEQ ID NO: 55) |
| Primer_P91F_F | GCGGGTATGCGTATCTTCATCATCAACGAACACGGT (SEQ ID NO: 56) |
| Primer_P91F_R | GATACGCATACCCGCCAGCGGACCGT (SEQ ID NO: 57) |
| Primer_L126F_F | GCCTGATCACCGACGCGTTCTGGTACTTCGCG (SEQ ID NO: 58) |
| Primer_L126F_R | CGCGTCGGTGATCAGGCAAGAAACTTCTTCGTC (SEQ ID NO: 59) |
| Primer_L126V_F | CTGATCACCGACGCGGTTTGGTACTTCGCGC (SEQ ID NO: 60) |
| Primer_L126V_R | CGCGTCGGTGATCAGGCAAGAAACTTCTTC (SEQ ID NO: 61) |
| Primer_N196Q_F | CAAATCTGCGTACTCTCAGTGGCAGATCCTGAAAGAAA (SEQ ID NO: 62) |
| Primer_N196Q_R | AGAGTACGCAGATTTGATGTCTTTAACTTTCAGCATCG (SEQ ID NO: 63) |
| Primer_I199F_F | GCGTACTCTAACTGGCAGTTCCTGAAAGAAATCCTGGG (SEQ ID NO: 64) |
| Primer_I199F_R | CTGCCAGTTAGAGTACGCAGATTTGATGTCTTTAAC (SEQ ID NO: 65) |
| Primer_I199L_F | GCGTACTCTAACTGGCAGCTGCTGAAAGAAATCCTGGG (SEQ ID NO: 66) |
| Primer_I199L_R | CTGCCAGTTAGAGTACGCAGATTTGATGTCTTT (SEQ ID NO: 67) |
| Primer_I199V_F | GCGTACTCTAACTGGCAGGTTCTGAAAGAAATCCTGGG (SEQ ID NO: 68) |
| Primer_I199V_R | CTGCCAGTTAGAGTACGCAGATTTGATGTCTTT (SEQ ID NO: 69) |
| Primer_L200I_F | TACTCTAACTGGCAGATCATCAAAGAAATCCTGGG (SEQ ID NO: 70) |
| Primer_L200I_R | CTGCCAGTTAGAGTACGCAGATTTGATGTCTTTAAC (SEQ ID NO: 71) |
| Primer_L200V_F | TACTCTAACTGGCAGATCGTTAAAGAAATCCTGGGTAA (SEQ ID NO: 72) |
| Primer_L200V_R | CTGCCAGTTAGAGTACGCAGATTTGATGTCTTTAAC (SEQ ID NO: 73) |
| Primer_I203L_F | GGCAGATCCTGAAAGAACTGCTGGGTAAAATGATCAAACAG (SEQ ID NO: 74) |
| Primer_I203L_R | TTCTTTCAGGATCTGCCAGTTAGAGTACGCAGATTTG (SEQ ID NO: 75) |
| Primer_I203V_F | GGCAGATCCTGAAAGAAGTTCTGGGTAAAATGATCAAACAGACC (SEQ ID NO: 76) |
| Primer_I203V_R | TTCTTTCAGGATCTGCCAGTTAGAGTACGCAGATTTG (SEQ ID NO: 77) |
| Primer_L204F_F | GGCAGATCCTGAAAGAAATCTTCGGTAAAATGATCAAACAGACC (SEQ ID NO: 78) |
| Primer_L204F_R | CTTTCAGGATCTGCCAGTTAGAGTACGCAG (SEQ ID NO: 79) |
| Primer_L204W_F | GATCCTGAAAGAAATCTGGGGTAAAATGATCAAACAGACC (SEQ ID NO: 80) |
| Primer_L204W_R | GATTTCTTTCAGGATCTGCCAGTTAGAGTACGCAG (SEQ ID NO: 81) |
| Primer_L379F_F | CTTCTCTGACTTCGGTTTCGACCAGCCGCTGAACG (SEQ ID NO: 82) |
| Primer_L379F_R | ACCGAAGTCAGAGAAGATCATCGGAACACCTTCGC (SEQ ID NO: 83) |
| Primer_L379I_F | CTTCTCTGACTTCGGTATCGACCAGCCGCTGAACG (SEQ ID NO: 84) |
| Primer_L379I_R | ACCGAAGTCAGAGAAGATCATCGGAACACCTTC (SEQ ID NO: 85) |
| Primer_L379V_F | CTTCTCTGACTTCGGTGTTGACCAGCCGCTGAACG (SEQ ID NO: 86) |
| Primer_L379V_R | ACCGAAGTCAGAGAAGATCATCGGAACACC (SEQ ID NO: 87) |
| Primer_L379W_F | CTTCTCTGACTTCGGTTGGGACCAGCCGCTGAACG (SEQ ID NO: 88) |
| Primer_L379W_R | ACCGAAGTCAGAGAAGATCATCGGAACACCTTCGC (SEQ ID NO: 89) |
| Primer_3A_F | ACTCTAACTGGCAGGCGGCGAAAGAAGCGCTGGGTAAAATGATCA (SEQ ID NO: 90) |
| Primer_3A_R | CGCCGCCTGCCAGTTAGAGTACGCAGATTTGATGTC (SEQ ID NO: 91) |
| Primer_4A_F1 | GTACTCTAACTGGCAGGCGGCGAAAGAAGCGGCGGGTAAA (SEQ ID NO: 92) |
| Primer_4A_R | ATGATCAAACAGACCAAAGCGCCGCCTGCCAGTTAGAGTACGCAGATTTGATGTCTTTAAC |
| | (SEQ ID NO: 93) |

2. Mutant Protein Expression and Purification

The expression vector containing the mutant that was verified to be correct was transformed into E. coli BL21(DE3). BL21 (DE3) was cultured overnight and transferred to 1 L LB (Amp=100 μg/mL) at 1% (v/v) and cultured at 37° C. and 200 rpm for 1 to 2h, then continued to culture at 16° C. and 160 rpm until OD600 is about 1.0. The final concentration of 0.1 mM IPTG was used for induction, and the cells were collected after 18 to 20 hours of overnight culture. The cells were resuspended with buffer A [20 mm Tris HCl (pH 8.0), 100 mM NaCl], added with 1 mM phenylmethylsulfonyl fluoride (PMSF), 2 mM MgCl₂ and 5 μg/mL DNaseI and mixed well, than held on ice for 30 minutes. The cells were lysed by high-pressure cell crusher, and centrifuged at high speed (10000 rpm, 99 min). The supernatant was spin-incubated with 1 ml Ni-NTA (4° C., 1h), and then eluted with 6-10 column volumes of 25 mM imidazole. Finally, 1 mL of 250 mM imidazole was used to incubate at 4° C. for 10-30 minutes to elute the target protein. BSA method was used to determine the concentration of the target protein, which was stored in 50% glycerol at −20° C.

SDS-PAGE results of some mutants (l126V, L126F, L379F, L379W, L379V) upon expression and purification were shown in FIG. 15.

3. Functional Verification of Mutants In Vitro

The enzymic reaction system includes: 10 μg protein, 1.5 mM UDP-glucose, 250 μM glycosyl acceptor substrate and buffer (20 mM Tris-HCl (pH=8.0), 100 mM NaCl). The reaction of each mutant protein for the same substrate was repeated three times.

Reaction conditions: 37° C., 30 min. The reaction was quenched with an equal volume of methanol. After vigorous shaking, the reaction was centrifuged at 12000 rpm for 30 min. The supernatant was used for HPLC detection. Detection method: mobile phase A (acetonitrile)-mobile phase B (water) gradient elution. The peak area of the catalytic product of the mutant was calculated and compared with the peak area of the catalytic product of wild-type SrUGT76G1.

4. Functional Analysis of Mutants In Vitro

(1) Catalytic Activities of Mutants on Substrate Steviolmonoside.

As shown in FIG. 16, the activities of mutants L85V, I199F, I199L and L379I on substrate steviolmonoside increased by 36.96%, 102%, 34% and 20% respectively. The activity of wild type is 100 and the activities of the mutants relative to wild type were shown in the Vertical Coordinate. The activities of P91F, L126F, 1203V, L379F, 3A, 4A on substrate were decreased to 20%. G87F was almost completely inactivated, and activities of M88V, 190L, 190V, L126V, N196Q, L200I, L200V, I203L, L204F, L204W and L379V were also significantly decreased.

(2) Catalytic Activities of Mutants on Substrate Steviolbioside.

As shown in FIG. 17, for substrate steviolbioside, activities of mutants L85V, M88V, 190L, 190V, P91F, L126F, I199F, I199L, I199VL200I, I203L, I203V, L204F, L379F, L379I and L379V on the substrate were increased, among which M88V, I199F and L200I present significant increase by 1.38 times, 1.29 times and 1.65 times respectively. The activities of mutants G87F and 4A on substrate decreased to 3% and 14%. The activities of L204W, L379W and 3A also decreased significantly. The activity of wild type is 100 and the activities of the mutants relative to wild type were shown in the Vertical Coordinate.

(3) Catalytic Activity of Mutant on Substrate Rubusoside.

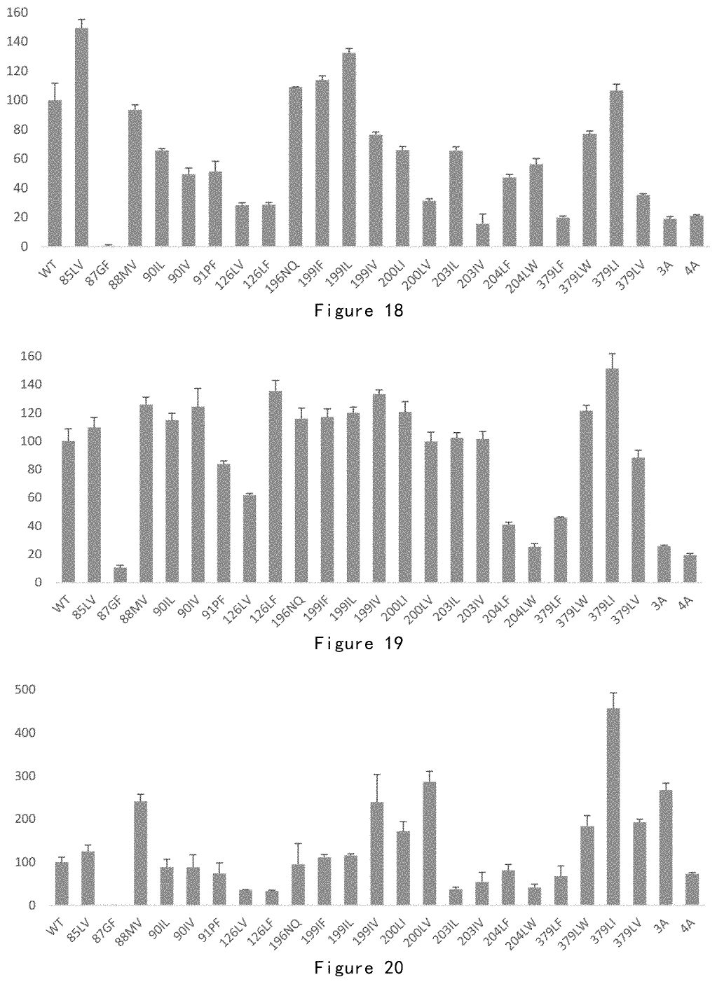

As shown in FIG. 18, for rubusoside, the activities of most mutants on the substrate decreased, and the activities of G87F, L126V, L126F, I203V, L379F, 3A and 4A decreased to 0.66%, 28%, 28%, 15%, 19%, 18% and 21% respectively. The activities of I90L, I90V, P91F, L200I, L200V, I203L, L204F, L204W and L379V also decreased significantly. However, mutants L85V, N196Q, I199F, I199L and L379I provide increased activity on the substrate, among which L85V and I199L were significant, 49% and 32% respectively. The activity of wild type is 100 and the activities of the mutants relative to wild type were shown in the Vertical Coordinate.

(4) Catalytic Activity of Mutant on Substrate Stevioside.

As shown in FIG. 19, the activities of the mutants on the substrate stevioside were changed. Among the mutants with enhanced activities, M88V, I90V, L126F, I199V, L200I, L379W and L379I increased significantly, which were 25%, 24%, 35%, 32%, 20%, 21% and 51% respectively. The activities of G87F, L204W, 3A and 4A decreased to 10%, 25%, 25% and 19% respectively. The activities of P91F, L126V, L204F, L379F and L379V also decreased significantly. The activity of wild type is 100 and the activities of the mutants relative to wild type were shown in the Vertical Coordinate.

(5) Catalytic Activity of Mutant on Substrate Rebaudioside A.

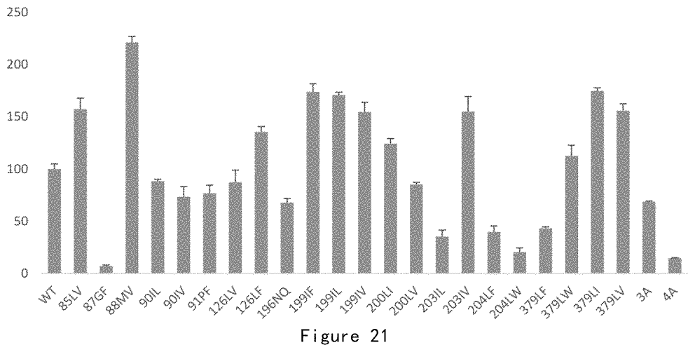

As shown in FIG. 20, the activities of mutants M88V, I199V, L200V, L379I and 3A on substrate rebaudioside A increased by 1.4 times, 1.39 times, 1.86 times, 3.57 times and 1.67 times respectively. The activities of L200I, L379V and L379W were also significantly improved. However, the activities of mutants G87F, L126V, L126F, I203L, I203V, L204W, L379F on substrate decreased. The activity of wild type is 100 and the activities of the mutants relative to wild type were shown in the Vertical Coordinate.

(6) Catalytic Activity of Mutant on Substrate Rebaudioside D.

As shown in FIG. 21, in vitro enzyme activity verification found that the activities of mutant L85V, M88V, L126F, I199F, I199L, I199V, L200I, I203V, L379W, L379I and L379V on substrate rebaudioside D increased by 57%, 121%, 35.6%, 73.7%, 70%, 54.6%, 24%, 55%, 12%, 74.6% and 55.9% respectively. The catalytic activities of mutants G87F, I203L, L204F, L204W, L379F and 4A decreased significantly, which were 7.25%, 35%, 39.8%, 20.5%, 43.3% and 14.6% respectively. N196Q also showed significantly decrease in activity. The activity of wild type is 100 and the activities of the mutants relative to wild type were shown in the Vertical Coordinate.

Each reference provided herein is incorporated by reference to the same extent as if each reference was individually incorporated by reference. In addition, it should be understood that based on the above teaching content of the disclosure, those skilled in the art can practice various changes or modifications to the disclosure, and these equivalent forms also fall within the scope of the appended claims.

1. A glycosyltransferase UGT76G1 mutant, wherein the mutant has a mutation in the amino acid interacting with the glycosyl donor or glycosyl receptor in its spatial structure and has changes in its catalytic activity, as compared to the wild-type glycosyltransferase UGT76G1.

2. The glycosyltransferase UGT76G1 mutant according to claim 1, wherein, the mutant is:
(a) a protein of amino acid sequence corresponds to SEQ ID NO: 1, with a mutation at residue 284, 147, 155, 146, 380, 85, 87, 88, 90, 91, 126, 196, 199, 200, 203, 204 or 379;
(b) a protein derived from (a) having one or more amino acids substituted, deleted, or inserted in the sequence, and having the function of the protein of (a), while the amino acids corresponding to residue 284, 147, 155, 146, 380, 85, 87, 88, 90, 91, 126, 196, 199, 200, 203, 204 or 379 of SEQ ID NO: 1 are the same as those mutated at the corresponding position of the protein of (a);
(c) a protein derived from (a) having more than 80% sequence identity with the amino acid sequence of the protein of (a), and having the function of the protein of (a), while the amino acids corresponding to residue 284, 147, 155, 146, 380, 85, 87, 88, 90, 91, 126, 196, 199, 200, 203, 204 or 379 of SEQ ID NO: 1 are the same as those mutated at the corresponding position of the protein of (a);
(d) the active fragment of the protein of (a), which contains the structure interacting with the glycosyl donor or glycosyl receptor in the spatial structure of glycosyltransferase UGT76G1, the amino acids corresponding to residue 284, 147, 155, 146, 380, 85, 87, 88, 90, 91, 126, 196, 199, 200, 203, 204 or 379 of SEQ ID NO: 1 are the same as those mutated at the corresponding position of the protein of (a).

3. The glycosyltransferase UGT76G1 mutant according to claim 2, wherein, the 284th residue is mutated to Ser and the catalytic activity of the mutant is increased, preferably, its catalytic activity for 1,3-glycosylation of a substrate containing 1,2-diglucosyl is increased or its catalytic activity for 1,3-glycosylation based on a monoglucosyl substrate is reduced; preferably, its catalytic activity for the substrate steviolbioside, stevioside or rebaudioside D is increased, while its catalytic activity for the substrate steviolmonoside, rubusoside and rebaudioside A is reduced; more preferably, its catalytic activity to convert rebaudioside D to rebaudioside M is increased and its catalytic activity to convert rebaudioside A to by-product rebaudioside I is decreased;
the 284th residue is mutated to Ala and the catalytic activity of the mutant is decreased;
the 147th residue is mutated to Ala, Asn or Gln, and the catalytic activity of the mutant is decreased;
the 155th residue is mutated to Ala or Tyr, and the catalytic activity of the mutant is decreased;
the 146th residue is mutated to Ala, Asn or Ser, and the catalytic activity of the mutant is decreased;
the 380th residue is mutated to Thr, Ser, Asn or Glu, and the catalytic activity of the mutant is decreased or eliminated;
the 85th residue is mutated to Val, and the catalytic activity of the mutant for steviolmonoside, steviolbioside, rubusoside or rebaudioside D is increased;
the 87th residue is mutated to Phe, and the catalytic activity of the mutant for steviolmonoside, steviolbioside, rubusoside, stevioside, rebaudioside A or rebaudioside D is decreased;
the 88th residue is mutated to Val, and the catalytic activity of the mutant for substrate steviolbioside, stevioside, rebaudioside A or rebaudioside D is increased, and the catalytic activity for substrate steviolmonoside is decreased;
the 90th residue is mutated to Leu, and the catalytic activity of the mutant for substrate steviolbioside is increased; the catalytic activity for substrate steviolmonoside or rubusoside is decreased;
the 90th residue is mutated to Val, and the catalytic activity of the mutant for substrate steviolbioside or stevioside is increased; the catalytic activity for substrate steviolmonoside or rubusoside is decreased;
the 91th residue is mutated to Phe, and the catalytic activity of the mutant for substrate steviolbioside is increased; the catalytic activity for substrate steviolmonoside, rubusoside or stevioside is decreased;
the 126th residue is mutated to Phe, and the catalytic activity of the mutant for substrate steviolbioside, stevioside or rebaudioside D is increased; the catalytic activity for substrate steviolmonoside, rubusoside or rebaudioside A is decreased;
the 126th residue is mutated to Val, and the catalytic activity of the mutant for substrate steviolmonoside, rubusoside, stevioside or rebaudioside A is decreased;
the 196th residue is mutated to Gln, and the catalytic activity of the mutant for substrate steviolmonoside or rebaudioside D is decreased;
the 199th residue is mutated to Phe, and the catalytic activity of the mutant for substrate steviolmonoside, steviolbioside or rebaudioside D is increased;
the 199th residue is mutated to Leu, and the catalytic activity of the mutant for substrate steviolmonoside, steviolbioside, rubusoside or rebaudioside D is increased.
the 199th residue is mutated to Val, and the catalytic activity of the mutant for substrate steviolbioside, stevioside, rebaudioside A or rebaudioside D is increased;
the 200th residue is mutated to Ile, and the catalytic activity of the mutant for substrate steviolbioside, rebaudioside A or rebaudioside D is increased; the catalytic activity for substrate steviolmonoside or rubusoside is decreased;
the 200th residue is mutated to Val, and the catalytic activity of the mutant for rebaudioside A is increased; the catalytic activity for substrate steviolmonoside or rubusoside is decreased;
the 203th residue is mutated to Leu, and the catalytic activity of the mutant for substrate steviolmonoside, rubusoside, rebaudioside A or rebaudioside D is decreased;
the 203th residue is mutated to Val, and the catalytic activity of the mutant for steviolbioside or rebaudioside D is increased; the catalytic activity for substrate steviolmonoside, rubusoside or rebaudioside A is decreased;
the 204th residue is mutated to Phe, and the catalytic activity of the mutant for substrate steviolmonoside, rubusoside, stevioside, or rebaudioside D is decreased;
the 204th residue is mutated to Trp, and the catalytic activity of the mutant for substrate steviolmonoside, steviolbioside, rubusoside, stevioside, rebaudioside A or rebaudioside D is decreased;
the 379th residue is mutated to Phe, and the catalytic activity of the mutant for substrate steviolbioside is increased; the catalytic activity for substrate steviolmonoside, rubusoside, stevioside or rebaudioside D is decreased;
the 379th residue is mutated to Ile, and the catalytic activity of the mutant for substrate steviolmonoside, steviolbioside, stevioside, rebaudioside A or rebaudioside D is increased;
the 379th residue is mutated to Val, and the catalytic activity of the mutant for substrate steviolbioside, rebaudioside A or rebaudioside D is increased; the catalytic activity for substrate steviolmonoside, rubusoside or stevioside is decreased;
the 379th residue is mutated to Trp, and the catalytic activity of the mutant for substrate rebaudioside A is increased; the catalytic activity for substrate steviolbioside is decreased;
the 199th, 200th, and 203th residues are mutated to Ala, and the catalytic activity of the mutant for substrate rebaudioside A is increased; the catalytic activity for substrate steviolmonoside, steviolbioside, rubusoside or stevioside is decreased; or
the 199th, 200th, 203th and 204th residues are mutated to Ala, and the catalytic activity of the mutant for substrate steviolmonoside, steviolbioside, rubusoside, stevioside or rebaudioside D is decreased.

4. An isolated polynucleotide, wherein the polynucleotide encodes the glycosyltransferase UGT76G1 mutant according to claim 1.

5. A vector, comprising the polynucleotide according to claim 4.

6. A genetically engineered host cell, comprising the vector according to claim 5.

7. The host cell according to claim 6, comprising: a reaction system for 1,3-glycosylation based on 1,2-diglucosyl or monoglucosyl substrate, wherein the enzyme for glycosylation is a glycosyltransferase UGT76G1 mutant; preferably, the reaction system is a system for rebaudioside M production.

8. The host cell according to claim 7, wherein the system for rebaudioside M production comprises:
a system with rebaudioside A as a substrate, including a glycosyltransferase UGT76G1 mutant with residue 284 mutated to Ser, residue 85 mutated to Val, residue 126 mutated to Phe, residue 199 mutated to Phe, residue 199 mutated to Leu or residue 203 mutated to Val, corresponding to SEQ ID NO: 1, and an enzyme for converting rebaudioside A into rebaudioside D; preferably, the enzyme for converting rebaudioside A into rebaudioside D includes: EUGT11, UGT91D2; or
a system with stevioside as a substrate, including an enzyme for converting stevioside to rebaudioside A, a glycosyltransferase UGT76G1 mutant with residue 284 mutated to Ser, residue 88 mutated to Val, residue 90 mutated to Val, residue 126 mutated to Phe, residue 199 mutated to Val, or residue 379 mutated to Ile, corresponding to SEQ ID NO: 1, and an enzyme for converting rebaudioside A into rebaudioside D; preferably, the enzyme for converting stevioside to rebaudioside A is also UGT76G1, UGT76G1 mutant, the enzyme for converting rebaudioside A into rebaudioside D includes: EUGT11, UGT91D2; or
a system with rebaudioside D as a substrate, including a glycosyltransferase UGT76G1 mutant with residue 284 mutated to Ser, residue 85 mutated to Val, residue 88 mutated to Val, residue 126 mutated to Phe, residue 199 mutated to Phe, residue 199 mutated to Leu, residue 199 mutated to Val, residue 200 mutated to Ile, residue 203 mutated to Val, residue 379 mutated to Ile, residue 379 mutated to Val, or residue 379 mutated to Trp, corresponding to SEQ ID NO: 1; or
a system with steviol as a substrate, including a glycosyltransferase UGT76G1 mutant with residue 284 mutated to Ser, residue 88 mutated to Val, residue 90 mutated to Val, residue 126 mutated to Phe, residue 199 mutated to Val, or residue 379 mutated to Be, corresponding to SEQ ID NO: 1, and an enzyme for converting rebaudioside A or stevioside into rebaudioside D and an enzyme for converting steviol into stevioside or rebaudioside A; the enzyme for converting steviol into stevioside or rebaudioside A includes: EUGT11, UGT91D2, UGT74G1, UGT85C2, UGT75L20, UGT75L21, UGT75W2, UGT75T4, UGT85A57, UGT85A58, UGT76G1, UGT76G1 mutant.

9. The host cell according to claim 6, wherein the host cell also includes an enzyme for recycling UDP glucose; preferably, the enzyme for recycling UDP glucose includes: AtSUS3.

10. The host cell according to claim 6, wherein the host cell include a prokaryotic cell or a eukaryotic cell; preferably, the prokaryotic cell includes Escherichia coli or Bacillus subtilis, the eukaryotic cell includes a fungal cell, a yeast cell, an insect cell or a mammalian cell.

11. A method for preparing the glycosyltransferase UGT76G1 mutant according to claim 1, comprising the steps of:
(1) culturing the host cell according to claim 6 to obtain a culture; and
(2) isolating the glycosyltransferase UGT76G1 mutant according to claim 1 from the culture.

12. A method of regulating the catalytic activity or substrate specificity of glycosyltransferase UGT76G1, including: mutating the amino acid interacting with glycosyl donor or glycosyl receptor in its spatial structure; thereby changing its catalytic activity or substrate specificity.

13. The method according to claim 12, wherein, comprising: mutating the 284th residue corresponding to SEQ ID NO:1 to Ser, and the catalytic activity of the mutant for 1,3-glycosylation of a substrate containing 1,2-diglucosyl is increased or its catalytic activity for 1,3-glycosylation based on a monoglucosyl substrate is reduced; preferably, its catalytic activity to convert rebaudioside D to rebaudioside M is increased and its catalytic activity to convert rebaudioside A to by-product rebaudioside I is decreased;
mutating the 284th residue corresponding to SEQ ID NO: 1 to Ala to decrease the catalytic activity of the mutant; or
mutating the 147th residue corresponding to SEQ ID NO: 1 to Ala, Asn or Gln to decrease the catalytic activity of the mutant;
mutating the 155th residue corresponding to SEQ ID NO: 1 to Ala or Tyr to decrease the catalytic activity of the mutant;
mutating the 146th residue corresponding to SEQ ID NO: 1 to Ala, Asn or Ser to decrease the catalytic activity of the mutant;
mutating the 380th residue corresponding to SEQ ID NO: 1 to Thr, Ser, Asn or Glu to decrease the catalytic activity of the mutant or eliminate the catalytic activity;
mutating the 85th residue corresponding to SEQ ID NO:1 to Val, and the catalytic activity of the mutant for substrate steviolmonoside, steviolbioside, rubusoside or rebaudioside D is increased;
mutating the 87th residue corresponding to SEQ ID NO:1 to Phe, and the catalytic activity of the mutant for substrate steviolmonoside, steviolbioside, rubusoside, stevioside, rebaudioside A or rebaudioside D is decreased;
mutating the 88th residue corresponding to SEQ ID NO:1 to Val, and the catalytic activity of the mutant for substrate steviolbioside, stevioside, rebaudioside A or rebaudioside D is increased, and its catalytic activity for substrate steviolmonoside is decreased;
mutating the 90th residue corresponding to SEQ ID NO:1 to Leu, and the catalytic activity of the mutant for substrate steviolbioside is increased, and its catalytic activity for substrate steviolmonoside or rubusoside is decreased;
mutating the 90th residue corresponding to SEQ ID NO:1 to Val, and the catalytic activity of the mutant for substrate steviolbioside or stevioside is increased, and its catalytic activity for substrate steviolmonoside or rubusoside is decreased;
mutating the 91th residue corresponding to SEQ ID NO:1 to Phe, and the catalytic activity of the mutant for substrate steviolbioside is increased, and its catalytic activity for substrate steviolmonoside, rubusoside or stevioside is decreased;
mutating the 126th residue corresponding to SEQ ID NO:1 to Phe, and the catalytic activity of the mutant for substrate steviolbioside, stevioside or rebaudioside D is increased, and its catalytic activity for substrate steviolmonoside, rubusoside or rebaudioside A is decreased;
mutating the 126th residue corresponding to SEQ ID NO:1 to Val, and the catalytic activity of the mutant for substrate steviolmonoside, rubusoside, stevioside or rebaudioside A is decreased;
mutating the 196th residue corresponding to SEQ ID NO:1 to Gln, and the catalytic activity of the mutant for substrate steviolmonoside or rebaudioside D is decreased;
mutating the 199th residue corresponding to SEQ ID NO:1 to Phe, and the catalytic activity of the mutant for substrate steviolmonoside, steviolbioside or rebaudioside D is increased;
mutating the 199th residue corresponding to SEQ ID NO:1 to Leu, and the catalytic activity of the mutant for substrate steviolmonoside, steviolbioside, rubusoside or rebaudioside D is increased;
mutating the 199th residue corresponding to SEQ ID NO:1 to Val, and the catalytic activity of the mutant for substrate steviolbioside, stevioside, rebaudioside A or rebaudioside D is increased;
mutating the 200th residue corresponding to SEQ ID NO:1 to Ile, and the catalytic activity of the mutant for substrate steviolbioside, rebaudioside A or rebaudioside D is increased, and its catalytic activity for substrate steviolmonoside or rubusoside is decreased;
mutating the 200th residue corresponding to SEQ ID NO:1 to Val, and the catalytic activity of the mutant for substrate rebaudioside A is increased, and its catalytic activity for substrate steviolmonoside, or rubusoside is decreased;
mutating the 203th residue corresponding to SEQ ID NO:1 to Leu, and the catalytic activity of the mutant for substrate steviolmonoside, rubusoside, rebaudioside A or rebaudioside D is decreased;
mutating the 203th residue corresponding to SEQ ID NO:1 to Val, and the catalytic activity of the mutant for substrate steviolbioside or rebaudioside D is increased, and its catalytic activity for substrate steviolmonoside, rubusoside or rebaudioside A is decreased;
mutating the 204th residue corresponding to SEQ ID NO:1 to Phe, and the catalytic activity of the mutant for substrate steviolmonoside, rubusoside, stevioside, or rebaudioside D is decreased;
mutating the 204th residue corresponding to SEQ ID NO:1 to Trp, and the catalytic activity of the mutant for substrate steviolmonoside, steviolbioside, rubusoside, stevioside, rebaudioside A or rebaudioside D is decreased;
mutating the 379th residue corresponding to SEQ ID NO:1 to Phe, and the catalytic activity of the mutant for substrate steviolbioside is increased, and its catalytic activity for substrate steviolmonoside, rubusoside, stevioside or rebaudioside D is decreased;
mutating the 379th residue corresponding to SEQ ID NO:1 to Ile, and the catalytic activity of the mutant for substrate steviolmonoside, steviolbioside, stevioside, rebaudioside A or rebaudioside D is increased;
mutating the 379th residue corresponding to SEQ ID NO:1 to Val, and the catalytic activity of the mutant for substrate steviolbioside, rebaudioside A or rebaudioside D is increased, and its catalytic activity for substrate steviolmonoside, rubusoside or stevioside is decreased;
mutating the 379th residue corresponding to SEQ ID NO:1 to Trp, and the catalytic activity of the mutant for substrate stevioside or rebaudioside A is increased, and its catalytic activity for substrate steviolbioside is decreased;
mutating the 199th, 200th, 203th residue corresponding to SEQ ID NO:1 to Ala, and the catalytic activity of the mutant for substrate rebaudioside A is increased, and its catalytic activity for substrate steviolmonoside, steviolbioside, rubusoside or stevioside is decreased; or
mutating the 199th, 200th, 203th 204th residues corresponding to SEQ ID NO:1 to Ala, and the catalytic activity of the mutant for substrate steviolmonoside, steviolbioside, rubusoside, stevioside or rebaudioside D is decreased.

14. (canceled)

15. A method of regulating glycosylation, comprising:
promoting 1,3-glycosylation of a substrate containing 1,2-diglucosyl by conducting catalyzation via a glycosyltransferase UGT76G1 mutant having residue 284 mutated to Ser corresponding to SEQ ID NO: 1;
conducting catalyzation via a glycosyltransferase UGT76G1 mutant having residue 284 mutated to Ala corresponding to SEQ ID NO: 1 to decrease catalytic activity of glycosylation; or
conducting catalyzation via a glycosyltransferase UGT76G1 mutant having residue 147 mutated to Ala, Asn or Gln corresponding to SEQ ID NO: 1 to decrease catalytic activity of glycosylation;
conducting catalyzation via a glycosyltransferase UGT76G1 mutant having residue 155 mutated to Ala or Tyr corresponding to SEQ ID NO: 1 to decrease catalytic activity of glycosylation;
conducting catalyzation via a glycosyltransferase UGT76G1 mutant having residue 146 mutated to Ala, Asn or Ser corresponding to SEQ ID NO: 1 to decrease catalytic activity of glycosylation;
conducting catalyzation via a glycosyltransferase UGT76G1 mutant having residue 380 mutated to Thr, Ser, Asn or Glu corresponding to SEQ ID NO: 1 to decrease glycosylation catalytic activity or eliminate the catalytic activity;
conducting catalyzation via a glycosyltransferase UGT76G1 mutant having residue 85 mutated to Val corresponding to SEQ ID NO: 1 to increase glycosylation catalytic activity for steviolmonoside, steviolbioside, rubusoside or rebaudioside D;
conducting catalyzation via a glycosyltransferase UGT76G1 mutant having residue 87 mutated to Phe corresponding to SEQ ID NO: 1 to decrease glycosylation catalytic activity for steviolmonoside, steviolbioside, rubusoside, stevioside, rebaudioside A or rebaudioside D;
conducting catalyzation via a glycosyltransferase UGT76G1 mutant having residue 88 mutated to Val corresponding to SEQ ID NO: 1 to increase glycosylation catalytic activity for substrate steviolbioside, stevioside, rebaudioside A or rebaudioside D; and to decrease glycosylation catalytic activity for substrate steviolmonoside;
conducting catalyzation via a glycosyltransferase UGT76G1 mutant having residue 90 mutated to Leu corresponding to SEQ ID NO: 1 to increase glycosylation catalytic activity for substrate steviolbioside; and to decrease glycosylation catalytic activity for substrate steviolmonoside or rubusoside;
conducting catalyzation via a glycosyltransferase UGT76G1 mutant having residue 90 mutated to Val corresponding to SEQ ID NO: 1 to increase glycosylation catalytic activity for substrate steviolbioside or stevioside; and to decrease glycosylation catalytic activity for substrate steviolmonoside or rubusoside;
conducting catalyzation via a glycosyltransferase UGT76G1 mutant having residue 91 mutated to Phe corresponding to SEQ ID NO: 1 to increase glycosylation catalytic activity for substrate steviolbioside; and to decrease glycosylation catalytic activity for substrate steviolmonoside, rubusoside, or stevioside;
conducting catalyzation via a glycosyltransferase UGT76G1 mutant having residue 126 mutated to Phe corresponding to SEQ ID NO: 1 to increase glycosylation catalytic activity for substrate steviolbioside, stevioside or rebaudioside D; and to decrease glycosylation catalytic activity for substrate steviolmonoside, rubusoside or rebaudioside A;
conducting catalyzation via a glycosyltransferase UGT76G1 mutant having residue 126 mutated to Val corresponding to SEQ ID NO: 1 to decrease glycosylation catalytic activity for steviolmonoside, rubusoside, stevioside or rebaudioside A;
conducting catalyzation via a glycosyltransferase UGT76G1 mutant having residue 196 mutated to Gln corresponding to SEQ ID NO: 1 to decrease glycosylation catalytic activity for steviolmonoside or rebaudioside D;
conducting catalyzation via a glycosyltransferase UGT76G1 mutant having residue 199 mutated to Phe corresponding to SEQ ID NO: 1 to increase glycosylation catalytic activity for steviolmonoside, steviolbioside or rebaudioside D;
conducting catalyzation via a glycosyltransferase UGT76G1 mutant having residue 199 mutated to Leu corresponding to SEQ ID NO: 1 to increase glycosylation catalytic activity for steviolmonoside, steviolbioside, rubusoside or rebaudioside D;
conducting catalyzation via a glycosyltransferase UGT76G1 mutant having residue 199 mutated to Val corresponding to SEQ ID NO: 1 to increase glycosylation catalytic activity for steviolbioside, stevioside, rebaudioside A or rebaudioside D;
conducting catalyzation via a glycosyltransferase UGT76G1 mutant having residue 200 mutated to Ile corresponding to SEQ ID NO: 1 to increase glycosylation catalytic activity for substrate steviolbioside, rebaudioside A or rebaudioside D; and to decrease glycosylation catalytic activity for substrate steviolmonoside or rubusoside;
conducting catalyzation via a glycosyltransferase UGT76G1 mutant having residue 200 mutated to Val corresponding to SEQ ID NO: 1 to increase glycosylation catalytic activity for substrate rebaudioside A; and to decrease glycosylation catalytic activity for substrate steviolmonoside or rubusoside;
conducting catalyzation via a glycosyltransferase UGT76G1 mutant having residue 203 mutated to Leu corresponding to SEQ ID NO: 1 to decrease glycosylation catalytic activity for substrate steviolmonoside, rubusoside, rebaudioside A or rebaudioside D;
conducting catalyzation via a glycosyltransferase UGT76G1 mutant having residue 203 mutated to Val corresponding to SEQ ID NO: 1 to increase glycosylation catalytic activity for substrate steviolbioside or rebaudioside D; and to decrease glycosylation catalytic activity for substrate steviolmonoside, rubusoside or rebaudioside A;
conducting catalyzation via a glycosyltransferase UGT76G1 mutant having residue 204 mutated to Phe corresponding to SEQ ID NO: 1 to decrease glycosylation catalytic activity for steviolmonoside, rubusoside, stevioside or rebaudioside D;
conducting catalyzation via a glycosyltransferase UGT76G1 mutant having residue 204 mutated to Trp corresponding to SEQ ID NO: 1 to decrease glycosylation catalytic activity for steviolmonoside, steviolbioside, rubusoside, stevioside, rebaudioside A or rebaudioside D;
conducting catalyzation via a glycosyltransferase UGT76G1 mutant having residue 379 mutated to Phe corresponding to SEQ ID NO: 1 to increase glycosylation catalytic activity for substrate steviolbioside; and to decrease glycosylation catalytic activity for substrate steviolmonoside, rubusoside, stevioside or rebaudioside D;
conducting catalyzation via a glycosyltransferase UGT76G1 mutant having residue 379 mutated to Ile corresponding to SEQ ID NO: 1 to increase glycosylation catalytic activity for steviolmonoside, steviolbioside, stevioside, rebaudioside A or rebaudioside D;
conducting catalyzation via a glycosyltransferase UGT76G1 mutant having residue 379 mutated to Val corresponding to SEQ ID NO: 1 to increase glycosylation catalytic activity for substrate steviolbioside, rebaudioside A or rebaudioside D; and to decrease glycosylation catalytic activity for substrate steviolmonoside, rubusoside or stevioside;
conducting catalyzation via a glycosyltransferase UGT76G1 mutant having residue 379 mutated to Trp corresponding to SEQ ID NO: 1 to increase glycosylation catalytic activity for substrate rebaudioside A; and to decrease glycosylation catalytic activity for substrate steviolbioside;
conducting catalyzation via a glycosyltransferase UGT76G1 mutant having residue 199, 200, 203 mutated to Ala corresponding to SEQ ID NO: 1 to increase glycosylation catalytic activity for substrate rebaudioside A; and to decrease glycosylation catalytic activity for substrate steviolmonoside, steviolbioside, rubusoside or stevioside; or
conducting catalyzation via a glycosyltransferase UGT76G1 mutant having residue 199, 200, 203, 204 mutated to Ala corresponding to SEQ ID NO: 1 to decrease glycosylation catalytic activity for steviolmonoside, steviolbioside, rubusoside, stevioside or rebaudioside D.

16. The method according to claim 15, wherein, the glycosylation product (1,3-glycosylation product) is rebaudioside M, and the method comprises:
conducting catalyzation with rebaudioside A as substrate via a glycosyltransferase UGT76G1 mutant with residue 284 mutated to Ser, residue 85 mutated to Val, residue 126 mutated to Phe, residue 199 mutated to Phe, residue 199 mutated to Leu or residue 203 mutated to Val, corresponding to SEQ ID NO: 1, and an enzyme for converting rebaudioside A into rebaudioside D, to produce rebaudioside M; preferably, the enzyme for converting rebaudioside A into rebaudioside D includes: EUGT11, UGT91D2; or
the method includes conducting catalyzation with stevioside as a substrate via an enzyme for converting stevioside to rebaudioside A, a glycosyltransferase UGT76G1 mutant with residue 284 mutated to Ser, residue 88 mutated to Val, residue 90 mutated to Val, residue 126 mutated to Phe, residue 199 mutated to Val, or residue 379 mutated to Be, corresponding to SEQ ID NO: 1, and an enzyme for converting rebaudioside A into rebaudioside D, to produce rebaudioside M; preferably, the enzyme for converting stevioside to rebaudioside A is also UGT76G1, UGT76G1 mutant, the enzyme for converting rebaudioside A into rebaudioside D includes: EUGT11, UGT91D2; or
the method includes conducting catalyzation with rebaudioside D as a substrate via a glycosyltransferase UGT76G1 mutant with residue 284 mutated to Ser, residue 85 mutated to Val, residue 88 mutated to Val, residue 126 mutated to Phe, residue 199 mutated to Phe, residue 199 mutated to Leu, residue 199 mutated to Val, residue 200 mutated to Ile, residue 203 mutated to Val, residue 379 mutated to Ile, residue 379 mutated to Val, or residue 379 mutated to Trp, corresponding to SEQ ID NO: 1, to produce rebaudioside M; or
the method includes conducting catalyzation with steviol as a substrate via a glycosyltransferase UGT76G1 mutant with residue 284 mutated to Ser, residue 88 mutated to Val, residue 90 mutated to Val, residue 126 mutated to Phe, residue 199 mutated to Val, or residue 379 mutated to Ile, corresponding to SEQ ID NO: 1, and an enzyme for converting rebaudioside A or stevioside into rebaudioside D and an enzyme for converting steviol into stevioside or rebaudioside A, to produce rebaudioside M; the enzyme for converting steviol into stevioside or rebaudioside A includes: EUGT11, UGT91D2, UGT74G1, UGT85C2, UGT75L20, UGT75L21, UGT75W2, UGT75T4, UGT85A57, UGT85A58, UGT76G1, UGT76G1 mutant.

17. The method according to claim 16, wherein, the method also comprises: using an enzyme for recycling UDP glucose; preferably, the enzyme for recycling UDP glucose includes: AtSUS3.

18. A composition comprising:
the glycosyltransferase UGT76G1 mutant according to claim 1.

19. A kit, comprising:
(i) the glycosyltransferase UGT76G1 mutant according to claim 1; or
(ii) a host cell comprising the polynucleotide encoding the glycosyltransferase UGT76G1 mutant of (i); or
(iii) a composition comprising the glycosyltransferase UGT76G1 mutant of (i).

20. A composition comprising the host cell according to claim 6.


SEQUENCE LISTING (95 sequences)

SEQ ID NO: 1 - protein, 458 aa, Stevia rebaudiana
MENKTETTVRRRRRIILFPVPFQGHINPILQLANVLYSKGFSITIFHTNFNKPKTSNYPHFTFRFILDNDPQDERISNLPTHGPLAGMRIPIINEHGADELRRELELLMLASEEDEEVSCLITDALWYFAQSVADSLNLRRLVLMTSSLFNFHAHVSLPQFDELGYLDPDDKTRLEEQASGFPMLKVKDIKSAYSNWQILKEILGKMIKQTKASSGVIWNSFKELEESELETVIREIPAPSFLIPLPKHLTASSSSLLDHDRTVFQWLDQQPPSSVLYVSFGSTSEVDEKDFLEIARGLVDSKQSFLWVVRPGFVKGSTWVEPLPDGFLGERGRIVKWVPQQEVLAHGAIGAFWTHSGWNSTLESVCEGVPMIFSDFGLDQPLNARYMSDVLKVGVYLENGWERGEIANAIRRVMVDEEGEYIRQNARVLKQKADVSLMKGGSSYESLESLVSYISSL

SEQ ID NO: 2 - protein, 458 aa, Artificial Sequence
  mutant T284S
MENKTETTVRRRRRIILFPVPFQGHINPILQLANVLYSKGFSITIFHTNFNKPKTSNYPHFTFRFILDNDPQDERISNLPTHGPLAGMRIPIINEHGADELRRELELLMLASEEDEEVSCLITDALWYFAQSVADSLNLRRLVLMTSSLFNFHAHVSLPQFDELGYLDPDDKTRLEEQASGFPMLKVKDIKSAYSNWQILKEILGKMIKQTKASSGVIWNSFKELEESELETVIREIPAPSFLIPLPKHLTASSSSLLDHDRTVFQWLDQQPPSSVLYVSFGSSSEVDEKDFLEIARGLVDSKQSFLWVVRPGFVKGSTWVEPLPDGFLGERGRIVKWVPQQEVLAHGAIGAFWTHSGWNSTLESVCEGVPMIFSDFGLDQPLNARYMSDVLKVGVYLENGWERGEIANAIRRVMVDEEGEYIRQNARVLKQKADVSLMKGGSSYESLESLVSYISSL

SEQ ID NO: 3 - protein, 462 aa, Artificial sequence
  EUGT11
MDSGYSSSYAAAAGMHVVICPWLAFGHLLPCLDLAQRLASRGHRVSFVSTPRNISRLPPVRPALAPLVAFVALPLPRVEGLPDGAESTNDVPHDRPDMVELHRRAFDGLAAPFSEFLGTACADWVIVDVFHHWAAAAALEHKVPCAMMLLGSAHMIASIADRRLERAETESPAAAGQGRPAAAPTFEVARMKLIRTKGSSGMSLAERFSLTLSRSSLVVGRSCVEFEPETVPLLSTLRGKPITFLGLMPPLHEGRREDGEDATVRWLDAQPAKSVVYVALGSEVPLGVEKVHELALGLELAGTRFLWALRKPTGVSDADLLPAGFEERTRGRGVVATRWVPQMSILAHAAVGAFLTHCGWNSTIEGLMFGHPLIMLPIFGDQGPNARLIEAKNAGLQVARNDGDGSFDREGVAAAIRAVAVEEESSKVFQAKAKKLQEIVADMACHERYIDGFIQQLRSYKD

SEQ ID NO: 4 - protein, 809 aa, Arabidopsis thaliana
MANPKLTRVLSTRDRVQDTLSAHRNELVALLSRYVDQGKGILQPHNLIDELESVIGDDETKKSLSDGPFGEILKSAMEAIVVPPFVALAVRPRPGVWEYVRVNVFELSVEQLTVSEYLRFKEELVDGPNSDPFCLELDFEPFNANVPRPSRSSSIGNGVQFLNRHLSSVMFRNKDCLEPLLDFLRVHKYKGHPLMLNDRIQSISRLQIQLSKAEDHISKLSQETPFSEFEYALQGMGFEKGWGDTAGRVLEMMHLLSDILQAPDPSSLEKFLGMVPMVFNVVILSPHGYFGQANVLGLPDTGGQVVYILDQVRALETEMLLRIKRQGLDISPSILIVTRLIPDAKGTTCNQRLERVSGTEHTHILRVPFRSEKGILRKWISRFDVWPYLENYAQDAASEIVGELQGVPDFIIGNYSDGNLVASLMAHRMGVTQCTIAHALEKTKYPDSDIYWKDFDNKYHFSCQFTADLIAMNNADFIITSTYQEIAGTKNTVGQYESHGAFTLPGLYRVVHGIDVFDPKFNIVSPGADMTIYFPYSEETRRLTALHGSIEEMLYSPDQTDEHVGTLSDRSKPILFSMARLDKVKNISGLVEMYSKNTKLRELVNLVVIAGNIDVNKSKDREEIVEIEKMHNLMKNYKLDGQFRWITAQTNRARNGELYRYIADTRGAFAQPAFYEAFGLTVVEAMTCGLPTFATCHGGPAEIIEHGLSGFHIDPYHPEQAGNIMADFFERCKEDPNHWKKVSDAGLQRIYERYTWKIYSERLMTLAGVYGFWKYVSKLERRETRRYLEMFYILKFRDLVKTVPSTADD

SEQ ID NO: 5 - protein, 473 aa, Stevia rebaudiana
MATSDSIVDDRKQLHVATFPWLAFGHILPYLQLSKLIAEKGHKVSFLSTTRNIQRLSSHISPLINVVQLTLPRVQELPEDAEATTDVHPEDIPYLKKASDGLQPEVTRFLEQHSPDWIIYDYTHYWLPSIAASLGISRAHFSVTTPWAIAYMGPSADAMINGSDGRTTVEDLTTPPKWFPFPTKVCWRKHDLARLVPYKAPGISDGYRMGLVLKGSDCLLSKCYHEFGTQWLPLLETLHQVPVVPVGLLPPEIPGDEKDETWVSIKKWLDGKQKGSVVYVALGSEVLVSQTEVVELALGLELSGLPFVWAYRKPKGPAKSDSVELPDGFVERTRDRGLVWTSWAPQLRILSHESVCGFLTHCGSGSIVEGLMFGHPLIMLPIFGDQPLNARLLEDKQVGIEIPRNEEDGCLTKESVARSLRSVVVEKEGEIYKANARELSKIYNDTKVEKEYVSQFVDYLEKNARAVAIDHES

SEQ ID NO: 6 - DNA, 33 bp, Artificial sequence
  Primer
aagtttcttgcctgatcaccaacgcgctgtggt

SEQ ID NO: 7 - DNA, 33 bp, Artificial sequence
  Primer
gttggtgatcaggcaagaaacttcttcgtcttc

SEQ ID NO: 8 - DNA, 33 bp, Artificial sequence
  Primer
tcttctctgacttcggtctggaacagccgctga

SEQ ID NO: 9 - DNA, 33 bp, Artificial sequence
  Primer
ttccagaccgaagtcagagaagatcatcggaac

SEQ ID NO: 10 - DNA, 33 bp, Artificial sequence
  Primer
tcttctctgacttcggtctgaaccagccgctga

SEQ ID NO: 11 - DNA, 33 bp, Artificial sequence
  Primer
gttcagaccgaagtcagagaagatcatcggaac

SEQ ID NO: 12 - DNA, 33 bp, Artificial sequence
  Primer
tcttctctgacttcggtctgtctcagccgctga

SEQ ID NO: 13 - DNA, 33 bp, Artificial sequence
  Primer
agacagaccgaagtcagagaagatcatcggaac

SEQ ID NO: 14 - DNA, 33 bp, Artificial sequence
  Primer
tcttctctgacttcggtctgacccagccgctga

SEQ ID NO: 15 - DNA, 33 bp, Artificial sequence
  Primer
ggtcagaccgaagtcagagaagatcatcggaac

SEQ ID NO: 16 - DNA, 33 bp, Artificial sequence
  Primer
tcccggttccgttccagggtgcgatcaacccga

SEQ ID NO: 17 - DNA, 33 bp, Artificial sequence
  Primer
cgcaccctggaacggaaccgggaacaggatgat

SEQ ID NO: 18 - DNA, 33 bp, Artificial sequence
  Primer
gtcgtctggttctgatgaccgcgtctctgttca

SEQ ID NO: 19 - DNA, 33 bp, Artificial sequence
  Primer
cgcggtcatcagaaccagacgacgcaggttcag

SEQ ID NO: 20 - DNA, 33 bp, Artificial sequence
  Primer
gtcgtctggttctgatgaccaactctctgttca

SEQ ID NO: 21 - DNA, 33 bp, Artificial sequence
  Primer
gttggtcatcagaaccagacgacgcaggttcag

SEQ ID NO: 22 - DNA, 33 bp, Artificial sequence
  Primer
gtcgtctggttctgatgacccagtctctgttca

SEQ ID NO: 23 - DNA, 33 bp, Artificial sequence
  Primer
ctgggtcatcagaaccagacgacgcaggttcag

SEQ ID NO: 24 - DNA, 33 bp, Artificial sequence
  Primer
tgcgtcgtctggttctgatggcgtcttctctgt

SEQ ID NO: 25 - DNA, 33 bp, Artificial sequence
  Primer
cgccatcagaaccagacgacgcaggttcagaga

SEQ ID NO: 26 - DNA, 33 bp, Artificial sequence
  Primer
tgcgtcgtctggttctgatgaactcttctctgt

SEQ ID NO: 27 - DNA, 33 bp, Artificial sequence
  Primer
gttcatcagaaccagacgacgcaggttcagaga

SEQ ID NO: 28 - DNA, 33 bp, Artificial sequence
  Primer
tgcgtcgtctggttctgatgtcttcttctctgt

SEQ ID NO: 29 - DNA, 33 bp, Artificial sequence
  Primer
agacatcagaaccagacgacgcaggttcagaga

SEQ ID NO: 30 - DNA, 33 bp, Artificial sequence
  Primer
tgtacgtttctttcggttctgcgtctgaagttg

SEQ ID NO: 31 - DNA, 33 bp, Artificial sequence
  Primer
cgcagaaccgaaagaaacgtacagaacagaaga

SEQ ID NO: 32 - DNA, 33 bp, Artificial sequence
  Primer
tgtacgtttctttcggttcttcttctgaagttg

SEQ ID NO: 33 - DNA, 33 bp, Artificial sequence
  Primer
agaagaaccgaaagaaacgtacagaacagaaga

SEQ ID NO: 34 - DNA, 28 bp, Artificial sequence
  Primer
ttcaacttccacgcggcggtttctctgc

SEQ ID NO: 35 - DNA, 21 bp, Artificial sequence
  Primer
cgccgcgtggaagttgaacag

SEQ ID NO: 36 - DNA, 28 bp, Artificial sequence
  Primer
ttcaacttccacgcgtatgtttctctgc

SEQ ID NO: 37 - DNA, 21 bp, Artificial sequence
  Primer
atacgcgtggaagttgaacag

SEQ ID NO: 38 - DNA, 30 bp, Artificial sequence
  Primer
cgcggatccatggactccggctactcctcc

SEQ ID NO: 39 - DNA, 61 bp, Artificial sequence
  Primer
aagctttcaatccttgtaagatctcaattgccgcggatccatggactccggctactcctcc

SEQ ID NO: 40 - DNA, 37 bp, Artificial sequence
  Primer
ctcaattggatatcggccggccatggcaaaccctaag

SEQ ID NO: 41 - DNA, 36 bp, Artificial sequence
  Primer
tttaccagactcgagggtacctcagtcatcggcggt

SEQ ID NO: 42 - DNA, 20 bp, Artificial sequence
  Primer
ctcgagtctggtaaagaaac

SEQ ID NO: 43 - DNA, 23 bp, Artificial sequence
  Primer
attggtacctcagtcatcggcgg

SEQ ID NO: 44 - DNA, 39 bp, Artificial sequence
  Primer
ccgatgactgaggtaccaataattttgtttaactttaag

SEQ ID NO: 45 - DNA, 40 bp, Artificial sequence
  Primer
gtttctttaccagactcgagttacagagaagagatgtaag

SEQ ID NO: 46 - DNA, 33 bp, Artificial sequence
  Primer
ccgacccacggtccggttgcgggtatgcgtatc

SEQ ID NO: 47 - DNA, 30 bp, Artificial sequence
  Primer
cggaccgtgggtcggcaggttagagatacg

SEQ ID NO: 48 - DNA, 30 bp, Artificial sequence
  Primer
ggtccgctggcgttcatgcgtatcccgatc

SEQ ID NO: 49 - DNA, 24 bp, Artificial sequence
  Primer
gaacgccagcggaccgtgggtcgg

SEQ ID NO: 50 - DNA, 30 bp, Artificial sequence
  Primer
ggtccgctggcgggtgttcgtatcccgatc

SEQ ID NO: 51 - DNA, 22 bp, Artificial sequence
  Primer
acccgccagcggaccgtgggtc

SEQ ID NO: 52 - DNA, 34 bp, Artificial sequence
  Primer
ctggcgggtatgcgtctgccgatcatcaacgaac

SEQ ID NO: 53 - DNA, 24 bp, Artificial sequence
  Primer
acgcatacccgccagcggaccgtg

SEQ ID NO: 54 - DNA, 34 bp, Artificial sequence
  Primer
ctggcgggtatgcgtgttccgatcatcaacgaac

SEQ ID NO: 55 - DNA, 22 bp, Artificial sequence
  Primer
acgcatacccgccagcggaccg

SEQ ID NO: 56 - DNA, 36 bp, Artificial sequence
  Primer
gcgggtatgcgtatcttcatcatcaacgaacacggt

SEQ ID NO: 57 - DNA, 26 bp, Artificial sequence
  Primer
gatacgcatacccgccagcggaccgt

SEQ ID NO: 58 - DNA, 32 bp, Artificial sequence
  Primer
gcctgatcaccgacgcgttctggtacttcgcg

SEQ ID NO: 59 - DNA, 33 bp, Artificial sequence
  Primer
cgcgtcggtgatcaggcaagaaacttcttcgtc

SEQ ID NO: 60 - DNA, 31 bp, Artificial sequence
  Primer
ctgatcaccgacgcggtttggtacttcgcgc

SEQ ID NO: 61 - DNA, 30 bp, Artificial sequence
  Primer
cgcgtcggtgatcaggcaagaaacttcttc

SEQ ID NO: 62 - DNA, 38 bp, Artificial sequence
  Primer
caaatctgcgtactctcagtggcagatcctgaaagaaa

SEQ ID NO: 63 - DNA, 38 bp, Artificial sequence
  Primer
agagtacgcagatttgatgtctttaactttcagcatcg

SEQ ID NO: 64 - DNA, 38 bp, Artificial sequence
  Primer
gcgtactctaactggcagttcctgaaagaaatcctggg

SEQ ID NO: 65 - DNA, 36 bp, Artificial sequence
  Primer
ctgccagttagagtacgcagatttgatgtctttaac

SEQ ID NO: 66 - DNA, 38 bp, Artificial sequence
  Primer
gcgtactctaactggcagctgctgaaagaaatcctggg

SEQ ID NO: 67 - DNA, 33 bp, Artificial sequence
  Primer
ctgccagttagagtacgcagatttgatgtcttt

SEQ ID NO: 68 - DNA, 38 bp, Artificial sequence
  Primer
gcgtactctaactggcaggttctgaaagaaatcctggg

SEQ ID NO: 69 - DNA, 33 bp, Artificial sequence; identical to SEQ ID NO: 67
  Primer

SEQ ID NO: 70 - DNA, 35 bp, Artificial sequence
  Primer
tactctaactggcagatcatcaaagaaatcctggg

SEQ ID NO: 71 - DNA, 36 bp, Artificial sequence; identical to SEQ ID NO: 65
  Primer

SEQ ID NO: 72 - DNA, 38 bp, Artificial sequence
  Primer
tactctaactggcagatcgttaaagaaatcctgggtaa

SEQ ID NO: 73 - DNA, 36 bp, Artificial sequence; identical to SEQ ID NO: 65
  Primer

SEQ ID NO: 74 - DNA, 41 bp, Artificial sequence
  Primer
ggcagatcctgaaagaactgctgggtaaaatgatcaaacag

SEQ ID NO: 75 - DNA, 37 bp, Artificial sequence
  Primer
ttctttcaggatctgccagttagagtacgcagatttg

SEQ ID NO: 76 - DNA, 44 bp, Artificial sequence
  Primer
ggcagatcctgaaagaagttctgggtaaaatgatcaaacagacc

SEQ ID NO: 77 - DNA, 37 bp, Artificial sequence; identical to SEQ ID NO: 75
  Primer

SEQ ID NO: 78 - DNA, 44 bp, Artificial sequence
  Primer
ggcagatcctgaaagaaatcttcggtaaaatgatcaaacagacc

SEQ ID NO: 79 - DNA, 30 bp, Artificial sequence
  Primer
ctttcaggatctgccagttagagtacgcag

SEQ ID NO: 80 - DNA, 40 bp, Artificial sequence
  Primer
gatcctgaaagaaatctggggtaaaatgatcaaacagacc

SEQ ID NO: 81 - DNA, 35 bp, Artificial sequence
  Primer
gatttctttcaggatctgccagttagagtacgcag

SEQ ID NO: 82 - DNA, 35 bp, Artificial sequence
  Primer
cttctctgacttcggtttcgaccagccgctgaacg

SEQ ID NO: 83 - DNA, 35 bp, Artificial sequence
  Primer
accgaagtcagagaagatcatcggaacaccttcgc

SEQ ID NO: 84 - DNA, 35 bp, Artificial sequence
  Primer
cttctctgacttcggtatcgaccagccgctgaacg

SEQ ID NO: 85 - DNA, 33 bp, Artificial sequence
  Primer
accgaagtcagagaagatcatcggaacaccttc

SEQ ID NO: 86 - DNA, 35 bp, Artificial sequence
  Primer
cttctctgacttcggtgttgaccagccgctgaacg

SEQ ID NO: 87 - DNA, 30 bp, Artificial sequence
  Primer
accgaagtcagagaagatcatcggaacacc

SEQ ID NO: 88 - DNA, 35 bp, Artificial sequence
  Primer
cttctctgacttcggttgggaccagccgctgaacg

SEQ ID NO: 89 - DNA, 35 bp, Artificial sequence; identical to SEQ ID NO: 83
  Primer

SEQ ID NO: 90 - DNA, 45 bp, Artificial sequence
  Primer
actctaactggcaggcggcgaaagaagcgctgggtaaaatgatca

SEQ ID NO: 91 - DNA, 36 bp, Artificial sequence
  Primer
cgccgcctgccagttagagtacgcagatttgatgtc

SEQ ID NO: 92 - DNA, 40 bp, Artificial sequence
  Primer
gtactctaactggcaggcggcgaaagaagcggcgggtaaa

SEQ ID NO: 93 - DNA, 61 bp, Artificial sequence
  Primer
atgatcaaacagaccaaagcgccgcctgccagttagagtacgcagatttgatgtctttaac

SEQ ID NO: 94 - DNA, 25 bp, Artificial sequence
  Primer
attctggatccatggaaaacaaaac

SEQ ID NO: 95 - DNA, 25 bp, Artificial sequence
  Primer
cgcaagcttttaactttacagagaa